SEQUENCE LISTING (30 sequences)

SEQ ID NO: 1 - protein, 456 aa, Arthrobacter aurescens
MILIRGLTRVITFDDQERELEDADILIDGPKIVAVGKDLSDRSVSRTIDGRGMIALPGLINSHQHLYEGAMRAIPQLERVTMASWLEGVLTRSAGWWRDGKFGPDVIREVARAVLLESLLGGITTVADQHLFFPGATADSYIDATIEAATDLGIRFHAARSSMTLGKSEGGFCDDLFVEPVDRVVQHCLGLIDQYHEPEPFGMVRIALGPCGVPYDKPELFEAFAQMAADYDVRLHTHFYEPLDAGMSDHLYGMTPWRFLEKHGWASDRVWLAHAVVPPREEIPEFADAGVAIAHLIAPDLRLGWGLAPIREYLDAGITVGFGTTGSASNDGGNLLGDLRLAALAHRPADPNEPEKWLSARELLRMATRGSAECLGRPDLGVLEEGRAADIACWRLDGVDRVGVHDPAIGLIMTGLSDRASLVVVNGQVLVENERPVLADLERIVANTTALIPKNL

SEQ ID NO: 2 - DNA, 1371 bp, Artificial Sequence
  Codon optimised open reading frame encoding TrzN
atgattctgatccgtggtctgacccgcgttatcaccttcgatgatcaggagcgtgaactggaagacgcggatattctgattgacggtccgaagattgtcgctgtgggtaaagatttaagcgatcgtagcgtgagccgcacgatcgatggtcgcggcatgattgccctgccgggtctgatcaatagccaccagcatctgtatgagggcgcgatgcgcgctattccgcaactggaacgcgttacgatggcgagctggttagagggcgtcctgactcgtagcgcgggttggtggcgtgacggtaaatttggtccggacgtcattcgcgaggtcgcacgtgccgtcctgctggaaagcctgctgggcggtatcaccaccgtcgccgatcagcatctgttttttccaggtgcaaccgcagatagctatattgatgcaaccatcgaagccgcgaccgacctgggtattcgctttcatgccgcgcgcagcagcatgactctgggtaagagcgaaggtggcttctgtgatgacctgtttgtggaaccagtggatcgtgtggttcagcattgcctgggcctgattgatcagtaccacgagccggaaccatttggcatggtccgcattgcactgggtccgtgcggcgttccgtatgataaaccagaactgtttgaggcgtttgcccagatggccgccgactacgacgtgcgcctgcatacgcatttttatgaaccgctggacgcgggtatgagcgatcatctgtatggcatgacgccgtggcgtttcttagagaagcatggctgggccagcgaccgcgtttggctggcacatgccgttgtgccgccgcgtgaagagattccagaatttgcggatgccggcgttgcaattgcccacctgattgcgccggatctgcgtctgggctggggtctggcaccgattcgtgaatatctggatgcgggcatcaccgtgggtttcggaaccaccggtagcgccagcaacgacggtggcaacctgttaggtgatctgcgcctggcagccctggcgcatcgcccggcggacccgaatgaaccagagaagtggctgagcgcccgtgaactgctgcgcatggctacgcgcggtagcgcagagtgtctgggccgcccggatctgggtgtgctggaagagggtcgtgccgcagacatcgcgtgctggcgcctggatggcgtggaccgcgtgggtgtgcacgatccggctatcggcctgatcatgaccggtctgagcgaccgcgcaagcctggttgtcgtcaacggccaggttctggtcgaaaatgaacgtccggttctggcggatctggaacgcatcgtggctaataccacggccctgattccgaaaaatctgtaa

SEQ ID NO: 3 - DNA, 1371 bp, Artificial Sequence
  Codon optimised open reading frame encoding TrzN with 156th codon change from TTT to TTC
atgattctgatccgtggtctgacccgcgttatcaccttcgatgatcaggagcgtgaactggaagacgcggatattctgattgacggtccgaagattgtcgctgtgggtaaagatttaagcgatcgtagcgtgagccgcacgatcgatggtcgcggcatgattgccctgccgggtctgatcaatagccaccagcatctgtatgagggcgcgatgcgcgctattccgcaactggaacgcgttacgatggcgagctggttagagggcgtcctgactcgtagcgcgggttggtggcgtgacggtaaatttggtccggacgtcattcgcgaggtcgcacgtgccgtcctgctggaaagcctgctgggcggtatcaccaccgtcgccgatcagcatctgttttttccaggtgcaaccgcagatagctatattgatgcaaccatcgaagccgcgaccgacctgggtattcgcttccatgccgcgcgcagcagcatgactctgggtaagagcgaaggtggcttctgtgatgacctgtttgtggaaccagtggatcgtgtggttcagcattgcctgggcctgattgatcagtaccacgagccggaaccatttggcatggtccgcattgcactgggtccgtgcggcgttccgtatgataaaccagaactgtttgaggcgtttgcccagatggccgccgactacgacgtgcgcctgcatacgcatttttatgaaccgctggacgcgggtatgagcgatcatctgtatggcatgacgccgtggcgtttcttagagaagcatggctgggccagcgaccgcgtttggctggcacatgccgttgtgccgccgcgtgaagagattccagaatttgcggatgccggcgttgcaattgcccacctgattgcgccggatctgcgtctgggctggggtctggcaccgattcgtgaatatctggatgcgggcatcaccgtgggtttcggaaccaccggtagcgccagcaacgacggtggcaacctgttaggtgatctgcgcctggcagccctggcgcatcgcccggcggacccgaatgaaccagagaagtggctgagcgcccgtgaactgctgcgcatggctacgcgcggtagcgcagagtgtctgggccgcccggatctgggtgtgctggaagagggtcgtgccgcagacatcgcgtgctggcgcctggatggcgtggaccgcgtgggtgtgcacgatccggctatcggcctgatcatgaccggtctgagcgaccgcgcaagcctggttgtcgtcaacggccaggttctggtcgaaaatgaacgtccggttctggcggatctggaacgcatcgtggctaataccacggccctgattccgaaaaatctgtaa

SEQ ID NO: 4 - DNA, 1371 bp, Arthrobacter aurescens
  misc_feature (792)..(792): n is a, c, g, or t
atgatcctgatccgcggactgacacgtgtcatcactttcgacgatcaagaacgcgagttggaagacgccgacattctgatcgacgggccgaagatcgtcgccgtcggaaaggacctgtcggatcgcagcgtttcacggacaatcgacgggcgtggaatgattgcgctgccaggtctcatcaactcgcaccagcacctgtacgaaggcgcaatgcgggcgattccccaactcgagcgggtcacgatggccagttggctcgaaggcgtcctgaccagaagcgcaggatggtggcgcgacgggaagttcggtccagatgtcatccgggaggttgcgcgcgccgtcctcctcgagtcgctcctcggtggcatcaccacggtcgccgaccagcacctattcttccccggtgccacggccgatagctatatcgacgcaaccatcgaggccgccaccgacctcgggatccggttccatgcggcaaggtcatcgatgacgctcggcaagagcgaaggtggcttctgcgacgacctgttcgtcgagcccgtcgacagagtcgtccagcactgtctgggtctcatcgaccagtaccacgagccggaaccgtttggaatggttcgaatcgctcttggaccctgcggcgtcccctacgacaagcccgaactcttcgaagcgttcgcacagatggcggcggactacgacgtgcgcctccacacccacttctacgaaccgctcgacgcgggaatgagcgaccacctctacggtatgacgccgtggcgtttccttgagaagcacggntgggccagcgaccgagtatggctggctcacgctgtggtacctccacgggaggagatccccgagttcgccgacgcaggcgtcgcgatcgcgcatctcattgcgccggaccttcggttgggctggggactcgcgcctatccgggaatacctcgacgccggaatcacggttgggtttggaacgaccgggtccgccagcaacgatggcgggaacctgcttggcgatctgcgcctggctgcgctggcgcaccgccccgctgaccccaacgagcccgaaaagtggctgtcggcacgcgagctgctccgaatggcaaccaggggatcagcggagtgcctcggcaggcccgacctgggcgtcctcgaggagggtcgtgctgcggacatcgcctgttggcgcctcgatggagtcgaccgggttggtgtgcacgacccggcgatcggcctgatcatgantggtctttcagaccgggcaagcctggtggtcgtgaatggtcaggtgctcgtcgagaacgaacgacccgttntggcagatntcgaacgcatcgtcgcgaatacgacagcactcattcccaagaacttgtag

SEQ ID NO: 5 - DNA, 42 bp, Artificial Sequence
  Oligonucleotide primer
ccacaacatatgattctgatccgtggtctgacccgcgttatc

SEQ ID NO: 6 - DNA, 45 bp, Artificial Sequence
  Oligonucleotide primer
cttcgaattcttacagatttttcggaatcagggccgtggtattag

SEQ ID NO: 7 - DNA, 72 bp, Artificial Sequence
  Oligonucleotide primer
cgcacgatcgatggtcgcggcatgattgccctgccgggtctgatcaatagccaccagcatctgnnsgagggc

SEQ ID NO: 8 - DNA, 62 bp, Artificial Sequence
  Oligonucleotide primer
cagatgctggtggctattgatcagacccggcagggcaatcatgccgcgaccatcgatcgtgc

SEQ ID NO: 9 - DNA, 48 bp, Artificial Sequence
  Oligonucleotide primer
gcgagctggttagagggcgtcctgnnscgtagcgcgggttggtggcgt

SEQ ID NO: 10 - DNA, 21 bp, Artificial Sequence
  Oligonucleotide primer
acgccaccaacccgcgctacg

SEQ ID NO: 11 - DNA, 73 bp, Artificial Sequence
  Oligonucleotide primer
tcctgctggaaagcctgctgggcggtatcaccaccgtcgccgatcagcatnnsttttttccaggtgcaaccgc

SEQ ID NO: 12 - DNA, 50 bp, Artificial Sequence
  Oligonucleotide primer
atgctgatcggcgacggtggtgataccgcccagcaggctttccagcagga

SEQ ID NO: 13 - DNA, 69 bp, Artificial Sequence
  Oligonucleotide primer
tatattgatgcaaccatcgaagccgcgaccgacctgggtattcgctttcatgccnnscgcagcagcatg

SEQ ID NO: 14 - DNA, 54 bp, Artificial Sequence
  Oligonucleotide primer
ggcatgaaagcgaatacccaggtcggtcgcggcttcgatggttgcatcaatata

SEQ ID NO: 15 - DNA, 69 bp, Artificial Sequence
  Oligonucleotide primer
accatcgaagccgcgaccgacctgggtattcgctttcatgccgcgcgcnnsagcatgactctgggtaag

SEQ ID NO: 16 - DNA, 48 bp, Artificial Sequence
  Oligonucleotide primer
gcgcgcggcatgaaagcgaatacccaggtcggtcgcggcttcgatggt

SEQ ID NO: 17 - DNA, 51 bp, Artificial Sequence
  Oligonucleotide primer
gaaccatttggcatggtccgcattgcactgggtnnstgcggcgttccgtat

SEQ ID NO: 18 - DNA, 33 bp, Artificial Sequence
  Oligonucleotide primer
acccagtgcaatgcggaccatgccaaatggttc

SEQ ID NO: 19 - DNA, 39 bp, Artificial Sequence
  Oligonucleotide primer
gtgcgcctgcatacgcatttttatgaaccgnnsgacgcg

SEQ ID NO: 20 - DNA, 30 bp, Artificial Sequence
  Oligonucleotide primer
cggttcataaaaatgcgtatgcaggcgcac

SEQ ID NO: 21 - DNA, 42 bp, Artificial Sequence
  Oligonucleotide primer
ctgcatacgcatttttatgaaccgctggacgcgnnsatgagc

SEQ ID NO: 22 - DNA, 33 bp, Artificial Sequence
  Oligonucleotide primer
cgcgtccagcggttcataaaaatgcgtatgcag

SEQ ID NO: 23 - DNA, 61 bp, Artificial Sequence
  Oligonucleotide primer
attccagaatttgcggatgccggcgttgcaattnnscacctgattgcgccggatctgcgtc

SEQ ID NO: 24 - DNA, 33 bp, Artificial Sequence
  Oligonucleotide primer
aattgcaacgccggcatccgcaaattctggaat

SEQ ID NO: 25 - DNA, 45 bp, Artificial Sequence
  Oligonucleotide primer
accggtagcgccagcaacgacggtggcaacnnsttaggtgatctg

SEQ ID NO: 26 - DNA, 30 bp, Artificial Sequence
  Oligonucleotide primer
gttgccaccgtcgttgctggcgctaccggt

SEQ ID NO: 27 - DNA, 54 bp, Artificial Sequence
  Oligonucleotide primer
ttaggtgatctgcgcctggcagccctggcgcatcgcccggcgnnsccgaatgaa

SEQ ID NO: 28 - DNA, 42 bp, Artificial Sequence
  Oligonucleotide primer
cgccgggcgatgcgccagggctgccaggcgcagatcacctaa

SEQ ID NO: 29 - DNA, 29 bp, Artificial Sequence
  Oligonucleotide primer
gctgtgggtaaannsttaagcgatcgtag

SEQ ID NO: 30 - DNA, 29 bp, Artificial Sequence
  Oligonucleotide primer
ctacgatcgcttaasnntttacccacagc

(19) United States
(12) Patent Application Publication
(10) Pub. No.: US 2011/0229450 A1
(43) Pub. Date: Sep. 22, 2011

(54) ENZYMES AND METHODS FOR DEGRADING S-TRIAZINES AND DIAZINES

(75) Inventors: Colin Scott, Melba, ACT (AU); Robyn J. Russell, Wanniassa (AU); Christopher W. Coppin, Ngunnawal, ACT (AU); John G. Oakeshott, Wanniassa, ACT (AU); Colin J. jackson, Grenoble (FR)

(73) Assignee: Commonwealth Scientific and industrial Research Organisation, Campbell, ACT (AU)

(21) Appl. No.: 13/061,754

(22) PCT Filed: Sep. 2, 2009
(86) PCT No.: PCT/AU2009/001136

§ 371 Date: Jun. 2, 2011

Related U.S. Application Data
(60) Provisional application No. 61/094,044, filed on Sep. 3, 2008.

(51) Int. Cl. A61K 38/46; C12N 9/14; C07H 21/00; C12N 15/63; C12N 5/10; C12N 1/21; C12N 1/19; A62D 3/02; A61P 39/02

(52) U.S. Cl. 424/94.6; 435/195; 536/23.2; 435/320.1; 435/419; 435/252.3; 435/254.2; 435/262.5

(57) ABSTRACT

The present invention relates to polypeptides for degrading s-triazines such as atrazine, as well as diazines. Also provided are polynucleotides encoding these polypeptides. The present invention also relates to the use of these polynucleotides and polypeptides in the bioremediation of s-triazines and diazines.

### FIELD OF THE INVENTION

The present invention relates to polypeptides for degrading s-triazines such as atrazine, as well as diazines. Also provided are polynucleotides encoding these polypeptides. The present invention also relates to the use of these polynucleotides and polypeptides in the bioremediation of s-triazines and diazines.

### BACKGROUND OF THE INVENTION

Current intensive farming practices are facilitated by the use of effective chemical pest control agents, such as the triazine herbicides. For example, atrazine (6-chloro-N²-ethyl-N⁴-isoproyl-1,3,5-triazine-2,4-diamine) is a highly-effective pre- and post emergence triazine herbicide that has been used extensively for the control of broadleaf weed species since it was first introduced in 1958 (Tomlin, 2006).

Atrazine at environmentally relevant concentrations has been causally linked to endocrine dysfunction in vertebrate species (demasculination of Xenopus laevis, for example) (Hayes et al., 2002, 2003 and 2006), and it has been suggested that atrazine may be carcinogenic (Huff, 2002; Huff and Sass, 2007). Additionally, due to their broad specificity, atrazine and related triazine herbicides have the potential to cause environmental damage via their toxic effects on non-target photosynthetic species.

Atrazine is both mobile and persistent in the environment. The environmental half life of atrazine has been estimated to be between four and fifty-seven weeks (Belluck et al., 1991), and atrazine has been detected in both surface and ground waters in several countries (Thurman and Meyer, 1996; van der Meer, 2006; Gavrilescu, 2005).

Several gene/enzyme systems have evolved in prokaryotes that allow the catabolism of the triazine pesticides as sources of carbon and nitrogen. The most thoroughly characterized of these pathways is encoded by the atzABCDEF genes from the transmissible pADP1 plasmid (Martinez et al., 2001) originally isolated from Pseudomonas sp. ADP (Mandelbaum et al., 1995; de Souza et al., 1995). Atrazine and simazine (6-chloro-N²,N⁴-diethyl-1,3,5-triazine-2,4-diamine) (de Souza et al., 1996) are successively dechlorinated and dealkylated by the amidohydrolase family enzymes encoded by atzA, atzB and atzC yielding cyanuric acid (de Souza et al., 1996; Boundy-Mills et al., 1997; Sadowsky et al., 1998), which is then mineralised to ammonia and carbon dioxide by the remaining hydrolases in the pathway, encoded by atzD, atzE and atzF (Fruchey et al., 2003; Cheng et al., 2005; Shapir et al., 2005a).

The AtzA enzyme of the AtzABCDEF atrazine degrading catabolic pathway is frequently replaced with the TrzN triazine-degrading enzyme (Sajjaphan et al., 2004), which has overlapping activities with AtzA (Shapir et al., 2005b), despite being only 25.4% identical. TrzN is a zinc-dependent amidohydrolase-family enzyme (Shapir et al., 2006), responsible for the hydrolytic displacement of chloride, fluoride, S-methyl, S(O)-methyl and cyano groups from triazine compounds (Shapir et al., 2005b). This means that TrzN targets s-triazines broadly, whilst AtzA can only be used to detoxify halogenated s-triazines. TrzN targets chloro-s-triazines (for example, atrazine, propazine and simazine), methyloxy-s-triazine (for example, atraton, simeton and prometon) and methylthio-s-triazine (for example, ametryn, prometryn and simetryn) herbicides.

TrzN is also reported to have a comparable catalytic constant to AtzA 2.1 sec⁻¹, compared with 5 sec⁻¹ for AtzA), but a much lower Kₘ for atrazine (20 μM compared to 100 μM for AtzA) (Shapir et al., 2006). AtzA therefore has a K_{cat}/Kₘ for atrazine of 3.3×10⁴, whilst TrzN has a K_{cat}/Kₘ of 1×10⁵ for atrazine, demonstrating TrzN to be a more catalytically efficient enzyme than AtzA.

However, unlike AtzA, TrzN has proven difficult to express in significant quantities in heterologous hosts, such as E. coli. A maximum yield of less than 10 mg.mL⁻¹ was obtained from E. coli when TrzN was coexpressed with the molecular chaperones GroEL (Shapir et al., 2006), with a maximum yield of only 560 μg.mL⁻¹ in the absence of the chaperones (Shapir et al., 2005b).

Bioremediation is an emerging approach to ameliorating the environmental impacts of potentially damaging pesticide residues (Alcalde et al., 2007). One successful bioremediation strategy is that of enzymatic bioremediation, where an isolated or semi-purified enzyme is used to catabolise or modify a toxic pesticide in such a way as to greatly reduce its toxicity (Parales et al., 2002; Sutherland et al., 2004). Enzymatic bioremediation has many advantages over the use of live microorganisms; there is release of GM organisms or intact DNA into the environment, the enzymes used are generally rapid (requiring an application time of only hours) and have a limited, predictable persistence after application (Alcalde et al., 2007).

However, the requirements of an enzyme to be employed in bioremediation are somewhat stringent, requiring a high catalytic activity, a low Kₘ, no dependence upon diffusible co-factors, and a generally robust protein toward a range of environmental conditions (pH, temperature, salt concentrations etc.). The enzyme must also be expressed as highly soluble, active protein in typical fermentation organisms, such as E. coli, and in this respect, TrzN is inadequate.

Although the potentially large environmental footprint of atrazine is concerning, it's continued use in agriculture is desirable. Therefore, there is a need for further methods for eliminating or reducing the potential of atrazine, and other s-triazines as well as diazines, for environmental damage.

### SUMMARY OF THE INVENTION

The present inventors have identified polynucleotides encoding TrzN, or variants thereof, with enhanced properties.

In a first aspect, the present invention provides an isolated and/or exogenous polynucleotide encoding a polypeptide which hydrolyses an s-triazine and/or diazine, wherein the polypeptide is at least 40% identical to a polypeptide comprising an amino acid sequence as provided in SEQ ID NO:1, and

i) when expressed in a bacterial cell more of the polypeptide is produced than by an isogenic bacterial cell cultured under identical conditions comprising an exogenous polynucleotide comprising a nucleotide sequence provided as SEQ ID NO:2 or SEQ ID NO:4, and/or

ii) the polypeptide has greater s-triazine and/or diazine hydrolysing activity than a polypeptide comprising an amino acid sequence as provided in SEQ ID NO:1.

In a preferred embodiment, more of the polypeptide is produced in soluble biologically active form than by an isogenic bacterial cell cultured under identical conditions comprising an exogenous polynucleotide comprising a nucleotide sequence provided as SEQ ID NO:2 or SEQ ID NO:4.

In a further particularly preferred embodiment, the polynucleotide encodes a polypeptide which comprises a threonine or valine at a position corresponding to amino acid number 159 of SEQ ID NO:1. In another embodiment, the polynucleotide additionally encodes a polypeptide which comprises i) an asparagine at a position corresponding to amino acid number 38 of SEQ ID NO:1, and ii) a proline, asparagine, threonine, aspartic acid, valine, glycine, cysteine, serine, glutamine, histidine, tyrosine or isoleucine at a position corresponding to amino acid number 131 of SEQ ID NO:1.

In a preferred embodiment, the polynucleotide comprises a nucleotide sequence provided as SEQ ID NO:2 or SEQ ID NO:4 with one or more of the following nucleotide substitutions, or a substitution at a nucleotide position corresponding thereto; T5C, C39A, C76A, C84A, T87C, C101A, T108A, T108A, G112A, A127G, C135T, A157T, C165T, G168A, C180T, C189T, A200T, C207T, G210A, G225T, A228G, C229T, T240C, A250C, C268A, G270A, A271T, T273A, C279T, A296G, A302G, A303G, A314G, C315A, T317C, T320C, A326C, A333G, T336C, C346T, G357A, A367G, C372T, C375A, C381T, T384C, C391A, C391G, C391T, T392C, T392A, T392G, G393C, T399C, C410T, C411A, C411T, A414G, A418C, T423C, T426A, A432T, C438T, C449G, C454T, T466C, T468C, T471C, C474T, G475A, C476T, A481G, C483T, G489A, G489T, T498C, T531A, A537G, A540G, A545G, T546G, G548A, T555C, T555C, C564T, G567A, G567A, G568A, G569A, G573C, T579C, A584T, G589A, A600G, T618C, T618C, T627C, C628G, G630A, C633T, G637A, T639C, T639C, A654G, G660A, G660T, T663C, C675A, G681T, C686T, C690A, C696T, G705A, A723G, C727G, T728C, T728G, G729C, G736A, G737C, G737A, G737T, T738G, T738C, T738C, G745A, G753C, G768A, T774A, C807A, T840A, A843G, A852T, A855G, C867T, T879C, G880A, G880T, G880C, C881T, G882T, C885T, G897A, T900C, T906A, A928G, A938T, T941C, C957A, T959A, C972T, T978A, C981T, C993T, C999T, C1003A, C1003T, T1011C, G1048A, G1048T, G1048C, A1049T, A1049G, G1053A, A1059G, A1086G, G1094A, T1101C, T1101G, C1128T, A1152G, G1176T, C1186A, C1186T, T1196C, C1203T, G1221A, C1223T, C1236T, G1248T, G1270A, C1278T, T1286A, T1305C, G1309A, C1321T, A1326G, C1329T, C1329T, C1332T, C1344A, C1351A and G1353T.

In an embodiment, the polynucleotide encodes a polypeptide comprising an amino acid sequence as provided in SEQ ID NO:1 with one or more of the following amino acid substitutions, or a substitution at an amino acid position corresponding thereto; I2T, F13L, L26M, D28E, A34D, D38N, S43G, M53L, Y67F, S84R, L90M, T91S, D99G, K101R, D105E, D105G, V106A, I107T, E109A, 1123V, L131P, L131N, L131T, L131D, L131V, L131G, L131C, L1315, L131Q, L131H, L131Y, L1311, T1371, 5140R, T1505, F156L, A159T, A159V, S161G, M1631, F177L, D182E, D182G, R183H, G190D, G1905, Y195F, E197K, P210A, V213I, M227I, M227I, A229V, D230E, L243P, L243G, G246A, G246S, G246D, G246E, G246K, G246V, D249N, A294T, A294S, A294L, 1310V, Y313F, L314P, V320E, L335M, D350N, D350Y, D350F, D350R, D350H, R365H, L396M, V399A, A408V, V424I, V429D, V437I and L451M.

In a further embodiment, the polynucleotide encodes a polypeptide comprising an amino acid sequence as provided in SEQ ID NO:1 with a substitution at one or more of the following amino acids, or an amino acid position corresponding thereto; M82, W85, L86, M92, L131, M163, L172, C211, Y215, H238, E241, L243, M247, H274, P299, D300, M303, W305, T325 and S329.

In another particularly preferred embodiment, the polynucleotide comprises a cytosine at a position corresponding to nucleotide number 468 of SEQ ID NO:2 or SEQ ID NO:4.

In a further preferred embodiment, the polynucleotide encodes a polypeptide which comprises;

i) a phenylalanine at a position corresponding to amino acid number 67 of SEQ ID NO:1, and/or

ii) a serine at a position corresponding to amino acid number 91 of SEQ ID NO:1, and/or

iii) a proline, asparagine, threonine, aspartic acid, valine, glycine, cysteine, serine, glutamine, histidine, tyrosine or isoleucine at a position corresponding to amino acid number 131 of SEQ ID NO:1, and/or

iv) a threonine or valine at a position corresponding to amino acid number 159 of SEQ ID NO:1, and/or

v) a glycine at a position corresponding to amino acid number 161 of SEQ ID NO:1, and/or

vi) an alanine at a position corresponding to amino acid number 210 of SEQ ID NO:1, and/or

vii) a proline or glycine at a position corresponding to amino acid number 243 of SEQ ID NO:1, and/or

viii) an aspartic acid, serine, glutamic acid, lysine, valine or alanine at a position corresponding to amino acid number 246 of SEQ ID NO:1, and/or

ix) a threonine, serine or leucine at a position corresponding to amino acid number 294 of SEQ ID NO:1, and/or

x) a methionine at a position corresponding to amino acid number 335 of SEQ ID NO:1, and/or

xi) a tyrosine, asparagine, phenylalanine, arginine or histidine at a position corresponding to amino acid number 350 of SEQ ID NO:1, and/or

xii) a biologically active fragment of any one of i) to xi).

In another embodiment, the polynucleotide encodes a polypeptide which comprises an amino acid sequence as provided in SEQ ID NO:1 with one of the following amino acid substitutions or groups of substitutions, or a substitution(s) at an amino acid position(s) corresponding thereto;

i) Y313F

ii) Y67F

iii) A159V

iv) A159V, L243P

v) D350Y

vi) G190D, M227I

vii) A159T

viii) A408V

ix) L26M, S161G

x) F13L, A34D, G246A, D350Y

xi) T137I, S140R

xii) L335M

xiii) P210A

xiv) A294T

xv) I123V

xvi) Y67F, V437I

xvii) M163I, D249N

xviii) T1371

xix) G246S

xx) L90M

xxi) A159V, L243P, L451M

xxii) T150S, A159V, A229V, D230E, L243P

xxiii) Y67F, L335M

xxiv) Y67F, K101R, A294T

xxv) L335M

xxvi) V106A, S161G, F177L, L335M

xxvii) S43G, 1107T, A159V, D350Y

xxviii) M53L, T137I, S140R, D182G, G190S, D350Y

xxix) A159V, L335M, D350Y

xxx) D28E, A294T, D350N

xxxi) P210A, V424I

xxxii) A159V, G190D

xxxiii) P210A, A294T, R365H, D350Y

xxxiv) 1123V, S161G, A294T

xxxv) Y67F, A159V, D350Y

xxxvi) T91S, A159V, A294T

xxxvii) Y67F, A159V, L243P

xxxviii) A159V, P210A

xxxix) A159V, I310V, L335M, L396M, L243P

xl) 12T, D105E, A159V, E197K, M227I, L243P, L335M

xli) A159V, L335M

xlii) S84R, D105G, A159V

xliii) Y67F, A294T

xliv) A159V, D182E, L335M, D350Y

xlv) Y67F, A159V, L243P

xlvi) D38N, A159V

xlvii) A159V, M163I, Y195F, D350Y

xlviii) F156L, P210A, D350Y

xlix) Y67F, D350Y

l) A159V, D350Y

li) Y67F, D99G, A159V, V213I, L243P, L335M

lii) E109A, A159V, L314P, V320E, V399A, V429D

liii) A159V, L335M

liv) Y67F, A159V, L335M, D350Y

lv) D38N, L131P, A159V

lvi) T91S, L131P, A159V, A294T, R365H, L396M, D350Y

lvii) R183H, P210A, D350Y

lviii) Y67F, A159V, D350Y

lix) A159V, P210A, A294T, D350N

lx) Y67F, A159V, D350N

lxi) A159V, L335M, D350Y

lxii) P210A, A294T, D350Y

lxiii) T91S, A159V, A294T

lxiv) P210A, A294T, L335M, or

lxv) Y67F, L335M.

In yet another embodiment, the polynucleotide comprises a nucleotide sequence provided as SEQ ID NO:2 or SEQ ID NO:4 with one of the following nucleotide substitutions or groups of substitutions, or a substitution(s) at an nucleotide position(s) corresponding thereto;

i) T468C,

ii) T468C, A938T,

iii) A200T, G210A, T468C,

iv) T468C, C476T, G753C,

v) T468C, C476T, T728C,

vi) T468C, 1048T,

vii) T384C, T468C, G569A, G681T,

viii) T468C, G475A,

ix) C279T, T468C, C1223T, C1329T,

x) C76A, T468C, A481G,

xi) C39A, C101A, T468C, T639C, G737C, G1048T,

xii) C410T, A418C, T468C, A600G,

xiii) T468C, G705A, C1003A,

xiv) T468C, G573C,

xv) T468C, C474T, C628G, C1278T,

xvi) T399C, T468C, G880A, T900C,

xvii) T468C, C1236T,

xviii) T87C, A367G, T468C,

xix) T468C, G1176T,

xx) C135T, T468C, C1344A,

xxi) T468C, A852T,

xxii) T468C, T738C,

xxiii) C454T, T468C,

xxiv) A200T, T468C, G1309A,

xxv) T468C, G489T, G745A,

xxvi) C410T, T468C,

xxvii) T468C, G736A,

xxviii) G225T, C268A, A414G, T468C, T627C, C1321T,

xxix) A432T, T468C, T471C, C476T, T728C, G1053A, C1351A,

xxx) A303G, C449G, T468C, C476T, C686T, C690A, T728C, C1128T,

xxxi) A200T, G210A, C372T, T468C, A654G, C1003A,

xxxii) C180T, A200T, A302G, C375A, T399C, C411T, T468C, A540G, G880A, T900C,

xxxiii) C229T, T468C, G705A, C1003A,

xxxiv) T317C, T468C, A481G, T531A, G753C, T906A, T978A, C1003A, A1326G,

xxxv) A127G, T320C, T468C, C476T, G753C, G1048T,

xxxvi) A157T, C410T, A418C, T468C, A545G, G568A, A600G, C628G, G630A, G1048T, C1332T,

xxxvii) T468C, C476T, A723G, C1003A, G1048T, C1329T,

xxxviii) C84A, T399C, T468C, C483T, G880A, T900C, G1048A,

xxxix) T468C, T498C, C628G, C885T, A1086G, G1270A,

xl) T384C, T468C, C476T, G569A, G753C, A1152G,

xli) T336C, T468C, C476T, A537G, C564T,

xlii) A228G, T240C, T468C, C628G, G880A, G1048T, G1094A,

xliii) T273A, A367G, C381T, T399C, T468C, A481G, G880A, T900C,

xliv) A200T, C207T, G210A, T468C, C476T, G1048T, A1059G,

xlv) A271T, A333G, T399C, T468C, C476T, A843G, G880A, T900C, C1236T,

xlvi) A200T, G210A, C346T, T468C, C476T, T579C, T728C, C1278T,

xlvii) C438T, T468C, C476T, A855G,

xlviii) G168A, T426A, T468C, C476T, C628G, C1203T, T1305C,

xlix) T468C, C476T, T663C, C1236T,

l) T384C, T468C, C476T, T728C, A928G, C1003A, C1186A,

li) TSC, C315A, T468C, C476T, G589A, G681T, T728C, G897A, C1003A,

lii) C279T, T468C, C476T, C1003A,

liii) A250C, A314G, T468C, C476T,

liv) A200T, G210A, T468C, T555C, G880A, T900C,

lv) T468C, C476T, T546G, C696T, C1003A, G1048T,

lvi) A200T, G210A, T468C, C476T, T728C, T1101G,

lvii) G112A, C165T, T468C, C476T, T618C, G753C, C999T, T1101C,

lviii) T423C, T468C, C476T, G489A, A584T, G753C, G1048T,

lix) T466C, T468C, C474T, C628G, G1048T,

lx) T108A, A200T, G210A, G357A, T468C, G1048T, A1059G, C1278T

lxi) T468C, C476T, G567A, G753C, G1048T,

lxii) A200T, G210A, A296G, T468C, C476T, G637A, T728C, C1003A,

lxiii) C189T, A326C, T468C, C476T, T941C, T959A, A1059G, C1186T, T1196C, G1248T, T1286A,

lxiv) T468C, C476T, C1003A,

lxv) A200T, G210A, C279T, T468C, C476T, T879C, C1003A, G1048T,

lxvi) G112A, C165T, T392C, T468C, C476T, T618C, G660A, C675A, G753C, C807A, C993T, C999T, T1101C, T1305C,

lxvii) A271T, T392C, T468C, C476T, G753C, G880A, G1048T, G1094A, C1186A, G1221A,

lxviii) A228G, T240C, T468C, G548A, C628G, G1048T, C1332T,

lxix) T108A, A200T, G210A, T423C, T468C, C476T, G567A, G1048T,

lxx) T384C, T468C, C476T, C628G, C867T, G880A, T900C, C981T, G1048A, A1326G,

lxxi) A200T, C207T, G210A, T468C, C476T, T840A, T900C, G1048A,

lxxii) T468C, C476T, C633T, G660T, A723G, C1003A, G1048T, C1329T,

lxxiii) A228G, T240C, G270A, T468C, C628G, 880A, G1048T,

lxxiv) A271T, A333G, T399C, T468C, C476T, A843G, G880A, T900C, C1003T, C1236T,

lxxv) A228G, T240C, T468C, C628G, G880A, C957A, C1003A, or

lxxvi) A200T, G210A, C411A, T468C, T555C, T639C, A654G, G768A, T774A, C972T, C1003A, T1011C, G1353T.

In an embodiment, when expressed in a bacterial cell at least twice, more preferably at least five times, the amount of the polypeptide is produced than by an isogenic bacterial cell cultured under identical conditions comprising an exogenous polynucleotide comprising a nucleotide sequence provided as SEQ ID NO:2 or SEQ ID NO:4.

In a further embodiment, when expressed in a bacterial cell at least twice, more preferably at least five times, the amount of soluble biologically active polypeptide is produced than by an isogenic bacterial cell cultured under identical conditions comprising an exogenous polynucleotide comprising a nucleotide sequence provided as SEQ ID NO:2 or SEQ ID NO:4.

In an alternate embodiment, when expressed in E. coli strain BL21 λDE3 and cultured on LB agar plates supplemented with 200 μg.mL⁻¹ ampicillin, 1 μM IPTG and impregnated with 1 mg.mL⁻¹ atrazine (90% atrazine w/w), clarification of the medium in the vicinity of colonies can be detected within about 8 days, more preferably within about 6 days, and even more preferably within about 2 days, of culturing. Expression of polynucleotides under such conditions is described in further detail in the Examples section.

In a preferred embodiment, the polypeptide has at least a two fold greater, more preferably at least a five fold greater, and even more preferably seven fold greater, atrazine hydrolysing activity than a polypeptide comprising an amino acid sequence as provided in SEQ ID NO:1.

In a preferred embodiment, the polypeptide has at least a two fold greater, more preferably at least a five fold greater, simazine hydrolysing activity than a polypeptide comprising an amino acid sequence as provided in SEQ ID NO:1.

Examples of s-triazines which can be hydrolysed be a polypeptide encoded by a polynucleotide of the invention include, but are not limited to, atrazine, ametryn, propazine, prometryn, simazine, simetryn, ipazine, trietazine or cyanozine.

The bacterial cell can be any cell which is capable of producing a polypeptide encoded by a polynucleotide of the invention. In a preferred embodiment, the bacterial cell is E. coli. Examples of suitable strains of E. coli include, but are not limited to, BL21 λDE3 (ATCC accession number PTA-2657), JM109 and DH10β.

In a further preferred embodiment, the polynucleotide is operably linked to a promoter capable of directing expression of the polynucleotide in a cell.

In another embodiment, the polynucleotide encodes a fusion protein which further comprises at least one other polypeptide sequence. The at least one other polypeptide may be, for example, a polypeptide that enhances the stability of a polypeptide of the present invention, a polypeptide that promotes the secretion of the fusion protein from a cell such as a bacterial cell or a yeast cell, or a polypeptide that assists in the purification of the fusion protein.

In another aspect, the present invention provides a vector comprising a polynucleotide of the invention.

Also provided is a host cell comprising a polynucleotide of the invention and/or a vector of the invention.

In an embodiment, the host cell further comprises an exogenous polynucleotide encoding a chaperone.

Examples of host cells of the invention include, but are not limited to, a bacterial cell, a yeast cell or a plant cell.

In a further aspect, the present invention provides a transgenic plant comprising at least one cell of the invention.

In yet another aspect, the present invention provides a transgenic non-human animal comprising at least one cell of the invention.

In a further aspect, the present invention provides a substantially purified and/or recombinant polypeptide which hydrolyses an s-triazine and/or diazine,

wherein the polypeptide is at least 40% identical to a polypeptide comprising an amino acid sequence as provided in SEQ ID NO:1, and wherein

i) when expressed in a bacterial cell more of the polypeptide is produced than by an isogenic bacterial cell cultured under identical conditions comprising an exogenous polynucleotide encoding the amino acid sequence provided as SEQ ID NO:1, and/or

ii) the polypeptide has greater s-triazine and/or diazine hydrolysing activity than a polypeptide comprising an amino acid sequence as provided in SEQ ID NO:1.

In a preferred embodiment, more of the polypeptide is produced in soluble biologically active form than by an isogenic bacterial cell cultured under identical conditions comprising an exogenous polynucleotide encoding the amino acid sequence provided as SEQ ID NO:1.

In a further particularly preferred embodiment, the polypeptide comprises a threonine or valine at a position corresponding to amino acid number 159 of SEQ ID NO: 1. In another embodiment, the polypeptide additionally comprises i) an asparagine at a position corresponding to amino acid number 38 of SEQ ID NO:1, and ii) a proline, asparagine, threonine, aspartic acid, valine, glycine, cysteine, serine, glutamine, histidine, tyrosine or isoleucine at a position corresponding to amino acid number 131 of SEQ ID NO:1.

Examples of s-triazines which can be hydrolysed be a polypeptide of the invention include, but are not limited to, atrazine, ametryn, propazine, prometryn, simazine, simetryn, ipazine, trietazine or cyanozine.

In an embodiment, the polypeptide is a fusion protein further comprises at least one other polypeptide sequence. The at least one other polypeptide may be, for example, a polypeptide that enhances the stability of a polypeptide of the present invention, a polypeptide that promotes the secretion of the fusion protein from a cell such as a bacterial cell or a yeast cell, or a polypeptide that assists in the purification of the fusion protein.

In another embodiment, the polypeptide is immobilized on a solid support.

In another aspect, the present invention provides an extract of a host cell of the invention, the plant of the invention and/or the animal of the invention, wherein the extract comprises a polypeptide of the invention.

In a further aspect, the present invention provides a composition comprising a polynucleotide of the invention, a vector of the invention, a host cell of the invention, a polypeptide of the invention and/or extract of the invention, and one or more acceptable carriers.

In a further aspect, the present invention provides method for hydrolysing an s-triazine or diazine, the method comprising contacting the s-triazine or diazine with a polynucleotide of the invention, a vector of the invention, a host cell of the invention, a polypeptide of the invention, an extract of the invention and/or a composition of the invention.

In an embodiment, the sample selected from the group consisting of: soil, water, biological material or a combination thereof.

In another aspect, the present invention provides a method of treating toxicity caused by an s-triazine or diazine in a subject, the method comprising administering to the subject a polynucleotide of the invention, a vector of the invention, a host cell of the invention, a polypeptide of the invention, an extract of the invention and/or a composition of the invention.

In another aspect, the present invention provides for the use of a polynucleotide of the invention, a vector of the invention, a host cell of the invention, a polypeptide of the invention, an extract of the invention and/or a composition of the invention for the manufacture of a medicament for treating toxicity caused by an s-triazine or diazine in a subject.

In another aspect, the present invention provides a method of producing a polypeptide capable of hydrolysing an s-triazine and/or diazine, the method comprising cultivating a host cell of the invention encoding said polypeptide, or a vector of the invention encoding said polypeptide, under conditions which allow expression of the polynucleotide encoding the polypeptide, and recovering the expressed polypeptide.

In yet a further aspect, the present invention provides a method for detecting a host cell, the method comprising

i) contacting a cell or a population of cells with a polynucleotide of the invention under conditions which allow uptake of the polynucleotide by the cell(s), and

ii) selecting a host cell by exposing the cells from step i), or progeny cells thereof, to a s-trizaine or a diazine.

In an embodiment, the polynucleotide encodes a polypeptide of the invention.

In an embodiment, the polynucleotide comprises a first open reading frame comprising a polynucleotide of the invention, and a second open reading frame not comprising a polynucleotide of the invention.

In one embodiment, the second open reading frame encodes a polypeptide. In a second embodiment, the second open reading frame encodes a polynucleotide which is not translated. In both instances, it is preferred that the second open reading frame is operably linked to a suitable promoter.

Preferably, the polynucleotide which is not translated encodes a catalytic nucleic acid, a dsRNA molecule, or an antisense molecule.

In a preferred embodiment, the cell is a plant cell.

In a further aspect, the present invention provides a kit for hydrolysing an s-triazine or diazine, the kit comprising a polynucleotide of the invention, a vector of the invention, a host cell of the invention, a polypeptide according of the invention, an extract of the invention and/or a composition of the invention.

In a further aspect, the present invention provides crystal of a polypeptide of the invention.

In another aspect, the present invention provides a method of designing a polypeptide which has greater s-triazine and/or diazine hydrolysing activity than a polypeptide comprising an amino acid sequence as provided in SEQ ID NO:1, the method comprising using the atomic coordinates of the crystal of the invention to computationally evaluate the ability of an s-triazine or diazine to associate with a candidate polypeptide, and selecting a polypeptide which has greater s-triazine and/or diazine hydrolysing activity than a polypeptide comprising an amino acid sequence as provided in SEQ ID NO:1.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Structure of various s-triazines degraded by enzymes of the invention.

FIG. 2. Schematic representation of pETcc2::egfp.

FIG. 3. Partial purification of wild-type TrzN, TrzN cc3.2 and the intermediate mutant forms. The value for wild-type TrzN is taken from Shapir et al. (2005) yielding 2.8 mg from 5 litres. Molecular weight markers were included (lane M), and their molecular weights (kDa) are indicated. The position of TrzN and its variants is indicated with an arrow. Equal volumes (5 μl) from identical purifications of TrzN and its variants were added to each lane.

FIG. 4. Schematic and cartoon of the TrzN structure. TrzN forms a homodimer of 99.6 kDa. Each monomer is divided into two domains: a β-sandwich domain and a β/α₈ barrel domain. The β/α₈ barrel has several loop insertions on the upper face, which serve to modify the active site entrance, or additionally form the dimer interface.

FIG. 5. Schematic showing the residues that constitute the substrate binding pocket of TrzNcc3.2. Atrazine, the Zn2+ centre and hydroxyl ion (OH⁻) are shown centrally. The identity and sequence position of the amino acids that comprise the substrate binding pocket are indicated.

FIG. 6. Saturation of Apo-TrzNcc3.2 with Zn²⁺. Half maximal activity was recovered at 2.6 μM ZnCl₂.

FIG. 7. Schematic of active site amino acid residues and metal coordinating amino acid residues. Atrazine, the Zn2⁺ and hydroxyl ion (OH⁻) are shown central and the identities of and sequence positions of the relevant residues are indicated.

FIG. 8. Schematic describing the proposed reaction mechanism of TrzN.

FIG. 9. Relationship between the rate constant (k_{cat}) of TrzN with ametryn and the pH of the reaction buffer.

FIG. 10. Result of field trials with TrzNcc3.2. Depletion of atrazine in a 1.5 mL holding dam at 10.5 hours after addition of TrzNcc3.2. Samples analysed by QHFSS (Squares), and CSIRO Entomology (Circles).

### KEY TO THE SEQUENCE LISTING

SEQ ID NO:1—Amino acid sequence of wild-type TrzN.
SEQ ID NO:2—Codon optimised open reading frame encoding TrzN (TrzNco).
SEQ ID NO:3—Codon optimised open reading frame encoding TrzN with 156^{th} codon change from TTT to TTC (Trz L1).
SEQ ID NO:4—Open reading frame encoding wild-type TrzN.
SEQ ID NO's 5 to 30—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, bioremediation, molecular genetics, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the terms “hydrolyses”, “hydrolysing”, “hydrolysing activity” and variations thereof refer to the ability of a polypeptide of the invention to catalyze the hydrolysis of a chemical bond. In a preferred embodiment, an enzyme of the invention is one or more of the following; a dehalogenase (for example, a chlorohydrolase, fluorohydrolase, halohydrolase, hydrolytic decholrinase, hydrolytic defluororinase and/or hydrolytic dehalogenase), a methoxyhydrolase and a methylthiohydrolase. In a preferred embodiment, the polypeptide “degrades” the s-triazine or diazine such that product of the activity of the enzyme is less toxic to, for example mammals and/or fish, and/or is less stable, than the s-triazine or diazine substrate.

As used herein, the term “greater s-triazine and/or diazine hydrolysing activity” refers to a polypeptide of the invention having a higher specific activity, catalytic constant (k_{cat}), substrate specificity (Kₘ) and/or second order rate constant (k_{cat}/Kₘ) for the s-triazine or diazine, or greater stability, than a polypeptide comprising the sequence of amino acids provided as SEQ ID NO:1. The specific activity can be determined as outlined in the Examples.

As used herein, the phrases “at an amino acid position corresponding thereto” and “at a position corresponding to amino acid number” refer to the relative position of the amino acid compared to surrounding amino acids. For example, in some embodiments a polypeptide of the invention may have deletional or substitutional mutations which alters the relative positioning of the amino acid when aligned against, for example, SEQ ID NO:1. In an embodiment, the polypeptide comprises the defined amino acid at the nominated residue number.

Similarly, the phrases “at a nucleotide position corresponding thereto” and “at a position corresponding to nucleotide number” refer to the relative position of the nucleotide compared to surrounding nucleotides. For example, in some embodiments a polynucleotide of the invention may have deletional or substitutional mutations which alters the relative positioning of the nucleotide when aligned against, for example, SEQ ID NO:2 or SEQ ID NO:4. In an embodiment, the polynucleotide comprises the defined nucleotide at the nominated nucleotide number.

As used herein the terms “treating”, “treat” or “treatment” include administering a therapeutically effective amount of a polypeptide of the invention, or a polynucleotide encoding therefor, sufficient to reduce or eliminate at least one symptom of toxicity caused by an s-triazine or diazine.

The term “biological material” is used herein in its broadest sense to include any product of biological origin. Such products include, but are not restricted to, food products for humans and animal feeds. The products include liquid media including water and liquid foodstuffs such as milk, as well as semi-solid foodstuffs such as yoghurt and the like. The present invention also extends to solid foodstuffs, particularly animal feeds. In an embodiment, it is preferred that the biological material is plant material such as, but not limited to, sugar cane, canola seeds, wheat seeds, barley seeds, sorghum seeds, rice, corn, pineapples, or cotton seeds.

As used herein, the term “extract” refers to any portion of a host cell, plant or non-human transgenic animal of the invention. The portion may be a whole entity such as a seed of a plant, or obtained by at least partial homogenization and/or purification. This term includes portions secreted from the host cell, and hence encompasses culture supernatants.

As used herein, the term “chaperone” refers to a protein whose function is to assist other proteins in achieving proper folding, or unfolding, for altering exportation of a protein from a cell. Chaperones are well known in the art, and include but are not limited to, ribosome binding proteins such as trigger factor (TF); the Hsp70 family of chaperones such as Hsp70, DnaK, Hsp40, DnaJ, GrpE and the Chaperonin family of chaperones such as GroEL, GroES, Hsp60, Hsp10. By way of a non-limiting example, the chaperone GroEL from E. coli is a member of the heat shock protein 60 (Hsp60) class of chaperones and is expressed, along with GroES, from the E. coli GroE operon. GroEL assists in protein folding reactions by binding unfolded proteins which decreases the concentration of aggregation-prone polypeptide intermediates and the rate of off-pathway aggregation, thereby favoring partitioning to the native conformation. It is known that the co-chaperonin GroES and cofactors such as ATP, K⁺ and Mg²⁺ further increase the yield of the GroEL mediated polypeptide folding reaction. Thus, in particular embodiments, the skilled artisan will include components, cofactors, additional chaperone proteins and the like, known in the art to improve or enhance chaperone mediated (or assisted) protein folding. Examples of vectors which can be used encoding GroEL include, but are not limited to, pG-KJE8 containing dnaK-dnaJ-grpE-groES-groEL, pGro7 containing groES-groEL and pG-Tf2 containing groES-groEL-tig (Nishihara et al., 1998 and 2000).

s-Triazines and Diazines

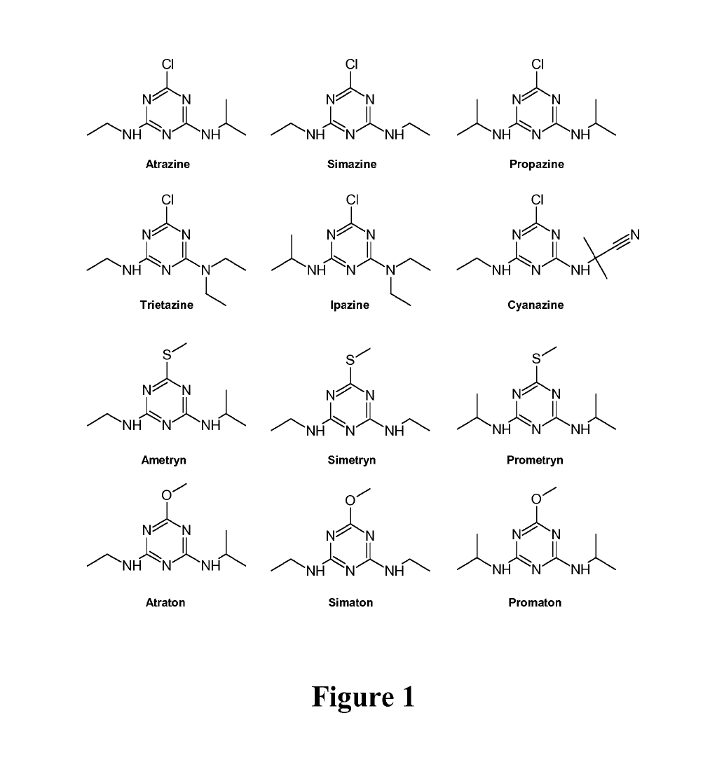

As used herein, an “s-triazine” is an organic chemical compound comprising a chemical structure having a six-membered heterocyclic aromatic ring consisting of three carbon atoms and three nitrogen atoms. The atoms in triazine rings are analogous to those in benzene rings. Examples of type of s-triazines hydrolysed (degraded) by the enzymes of the invention include, but are not limited to, chloro-s-triazines, fluoro-s-triazines, methylthio-s-triazines and methyloxy-s-triazines. The chemical structure of some s-triazines hydrolysed (degraded) by the enzymes of the invention are provided in FIG. 1. In a preferred embodiment, the s-triazine has the structure -2-R1-4-R2-6-R3-1,3,5-trainzine; where R1 can be Cl, Fl, OCH3, SCH3, S(O)CH3, or N3 where R2 or R3 can be OCH3, NHCH2CH2OH, NH(CH2)2CH3, NH(CH2)3CH3, NHCH2CH(CH3)2, NHCH(CH3(CH2CH3, NHC(CH3)2CN, NHC(CH3)3, NH2, or OH.

Chlorinated (chloro) s-triazines comprise at least one chloride. Examples of chlorinated s-triazines include, but are not limited to, atrazine (6-chloro-N-ethyl-N′-(1-methylethyl)-1,3,5-triazine-2,4-diamine) (see FIG. 1), chlorazine (6-chloro-N,N,N′,N′-tetraethyl-1,3,5-triazine-2,4-diamine), cyanazine (2-[[4-chloro-6-(ethyamino)-1,3,5-triazin-2-yl]amino]-2-methylpropanenitrile), cyprazine (6-chloro N-cyclopropyl-N′-(1-methylethyl)-1,3,5-triazine-2,4-diamine), eglinazine (N-[4-chloro-6-(ethylamino)-1,3,5-triazin-2-yl]glycine), ipazine (6-chloro-N,N-diethyl-N′-(1-methylethyl)-1,3,5-triazine-2,4-diamine), mesoprazine, (6-chloro-N-(3-methoxypropyl)-N′-(1-methylethyl)-1,3,5-triazine-2,4-diamine), procyazine (2-[[4-chloro-6-(cyclopropylamino)-1,3,5-triazin-2-yl]-amino]-2-methylpropanenitrile), proglinazine N-[4-chloro-6-[(1-methylethyl)amino]-1,3,5-triazin-2-yl]glycine), propazine (6-chloro-N,N′-bis(1-methylethyl)-1,3,5-triazine-2,4-diamine), sebuthylazine (6-chloro-N-ethyl-N′-(1-methylpropyl)-1,3,5-triazine-2,4-diamine, simazine (6-chloro-N,N″-diethyl-1,3,5-triazine-2,4-diamine), terbuthylazine (6-chloro-N-(1,1-dimethylethyl)-N′-ethyl-1,3,5-triazine-2,4-diamine) and trietazine (6-chloro-N,N,N′-triethyl-1,3,5-triazine-2,4-diamine), as well as products of chloro-s-triazine dealkylation (including atrazine dealkylation such as desethyl atrazine and desisopropyl atrazine).

Methylthio-s-triazines comprise at least one thiol group. Examples of methylthio-s-triazines include, but are not limited to, ametryn (N2-ethyl-N4-isopropyl-6-methylthio-1,3,5-triazine-2,4-diamine), prometryn (N2,N4-diisopropyl-6-methylthio-1,3,5-triazine-2,4-diamine) and simetryn (N2,N4-diethyl-6-methylthio-1,3,5-triazine-2,4-diamine).

Methoxy-s-triazines comprise at least one methoxy group. Examples of methoxy-s-triazines include, but are not limited to, atraton (N2-ethyl-N4-isopropyl-6-methoxy-1,3,5-triazine-2,4-diamine), simeton (N2,N4-diethyl-6-methoxy-1,3,5-triazine-2,4-diamine) and prometon (N2,N4-diisopropyl-6-methoxy-1,3,5-triazine-2,4-diamine).

Fluoro-s-triazines comprise at least one fluorine. An example of a fluoro-s-triazine is fluoratrazine (2-fluoro-4-N-ethylamino-6-N-isopropylamino-1,3,5-triazine).

In an embodiment, the s-triazine has at least one N-ethyl, N-isolpropyl, diethyl and/or N-cyanodimethylmethyl alkyl side chain.

In a preferred embodiment, the diazine has the structure -2-R1-4-R2-6-R3-1,3-pyrimidine, where R1 can be Cl, Fl, OCH3, SCH3, S(O)CH3, or N3 where R2 or R3 can be OCH3, NHCH2CH2OH, NH(CH2)2CH3, NH(CH2)3CH3, NHCH2CH(CH3)₂, NHCH(CH3)CH2CH3, NHC(CH3)2CN, NHC(CH3)3, NH2, or OH. Examples of types of diazines include, but are not limited to, chloro-s-diazines and fluoro-s-diazines. An example of a diazine which is a herbicide is Bromacil (5-bromo-3-sec-butyl-6-methyluracil).

### Polypeptides

By “substantially purified” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated. However, at present there is no evidence that the polypeptides of the invention exist in nature.

The term “recombinant” in the context of a polypeptide refers to the polypeptide when produced by a cell, or in a cell-free expression system, in an altered amount or at an altered rate compared to its native state. In one embodiment the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, biologically active fragments, modifications, analogous and/or derivatives of the polypeptides described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the query sequence is at least 400 amino acids in length and the GAP analysis aligns the two sequences over a region of at least 400 amino acids. Even more preferably, the GAP analysis aligns the two sequences over their entire length.

As used herein a “biologically active fragment” is a portion of a polypeptide as described herein which maintains a defined activity of the full-length polypeptide. Biologically active fragments can be any size as long as they maintain the defined activity. Preferably, biologically active fragments are at least 100, more preferably at least 400, amino acids in length.

A preferred embodiment relates to the polypeptide being produced in a “soluble biologically active form”. As the skilled addressee will appreciate, this refers to polypeptides which are not present in insoluble, and hence inactive, form when expressed in the cell. Furthermore, biologically active means the ability to hydrolyse an s-triazine and/or diazine.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 50%, more preferably at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of a polypeptide described herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide described herein can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they are able to confer the desired phenotype such as enhanced activity and/or altered substrate specificity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

In a preferred embodiment a mutant/variant polypeptide has one or two or three or four conservative amino acid changes when compared to a polypeptide specifically defined herein. Details of conservative amino acid changes are provided in Table 1.

In a preferred embodiment, the polypeptide comprises an amino acid sequence as provided in SEQ ID NO:1 with one or more of the following amino acid substitutions, or a substitution at an amino acid position corresponding thereto; 12T, F13L, L26M, D28E, A34D, D38N, S43G, M53L, Y67F, S84R, L90M, T91S, D99G, K101R, D105E, D105G, V106A, 1107T, E109A, 1123V, L131P, L131N, L131T, L131D, L131V, L131G, L131C, L1315, L131Q, L131H, L131Y, L1311, T1371, 5140R, T1505, F156L, A159T, A159V, S161G, M1631, F177L, D182E, D182G, R183H, G190D, G1905, Y195F, E197K, P210A, V213I, M227I, M227I, A229V, D230E, L243P, L243G, G246A, G246S, G246D, G246E, G246K, G246V, D249N, A294T, A294S, A294L, 1310V, Y313F, L314P, V320E, L335M, D350N, D350Y, D350F, D350R, D350H, R365H, L396M, V399A, A408V, V424I, V429D, V437I and L451M.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

In a further embodiment, the polypeptide comprises an amino acid sequence as provided in SEQ ID NO:1 with a substitution at one or more of the following amino acids, or an amino acid position corresponding thereto; M82, W85, L86, M92, L131, M163, L172, C211, Y215, H238, E241, L243, M247, H274, P299, D300, M303, W305, T325 and S329. One or more of these amino acids may changed to alter the specific activity, catalytic constant (k_{cat}), substrate specificity (Kₘ), stability and/or second order rate constant (k_{cat}/Kₘ) of the polypeptide.

In a further preferred embodiment, the polypeptide comprises;

i) a phenylalanine at a position corresponding to amino acid number 67 of SEQ ID NO:1, and/or

ii) a serine at a position corresponding to amino acid number 91 of SEQ ID NO:1, and/or

iii) a proline, asparagine, threonine, aspartic acid, valine, glycine, cysteine, serine, glutamine, histidine, tyrosine or isoleucine, at a position corresponding to amino acid number 131 of SEQ ID NO:1, and/or

iv) a threonine or valine at a position corresponding to amino acid number 159 of SEQ ID NO:1, and/or

v) a glycine at a position corresponding to amino acid number 161 of SEQ ID NO:1, and/or

vii) an alanine at a position corresponding to amino acid number 210 of SEQ ID NO:1, and/or

vii) a proline or glycine at a position corresponding to amino acid number 243 of SEQ ID NO:1, and/or

viii) an aspartic acid, serine, glutamic acid, lysine, valine or alanine at a position corresponding to amino acid number 246 of SEQ ID NO:1, and/or

ix) a threonine, serine or leucine at a position corresponding to amino acid number 294 of SEQ ID NO:1, and/or

x) a methionine at a position corresponding to amino acid number 335 of SEQ ID NO:1, and/or

xi) a tyrosine, asparagine, phenylalanine, arginine or histidine at a position corresponding to amino acid number 350 of SEQ ID NO:1, and/or

xii) a biologically active fragment of any one of i) to xi).

In an embodiment, the polypeptide comprises an amino acid sequence as provided in SEQ ID NO:1 with one of the following amino acid substitutions or groups of substitutions, or a substitution(s) at an amino acid position(s) corresponding thereto;

i) Y313F

ii) Y67F

iii) A159V

iv) A159V, L243P

v) D350Y

vi) G190D, M227I

vii) A159T

viii) A408V

ix) L26M, S161G

x) F13L, A34D, G246A, D350Y

xi) T1371, S140R

xii) L335M

xiii) P210A

xiv) A294T

xv) I123V

xvi) Y67F, V437I

xvii) M163I, D249N

xviii) T1371

xix) G246S

xx) L90M

xxi) A159V, L243P, L451M

xxii) T150S, A159V, A229V, D230E, L243P

xxiii) Y67F, L335M

xxiv) Y67F, K101R, A294T

xxv) L335M

xxvi) V106A, S161G, F177L, L335M

xxvii) S43G, 1107T, A159V, D350Y

xxviii) M53L, T137I, S140R, D182G, G190S, D350Y

xxix) A159V, L335M, D350Y

xxx) D28E, A294T, D350N

xxxi) P210A, V424I

xxxii) A159V, G190D

xxxiii) P210A, A294T, R365H, D350Y

xxxiv) 1123V, S161G, A294T

xxxv) Y67F, A159V, D350Y

xxxvi) T91S, A159V, A294T

xxxvii) Y67F, A159V, L243P

xxxviii) A159V, P210A

xxxix) A159V, 1310V, L335M, L396M, L243P

xl) 12T, D105E, A159V, E197K, M227I, L243P, L335M

xli) A159V, L335M

xlii) S84R, D105G, A159V

xliii) Y67F, A294T

xliv) A159V, D182E, L335M, D350Y

xlv) Y67F, A159V, L243P

xlvi) D38N, A159V

xlvii) A159V, M163I, Y195F, D350Y

xlviii) F156L, P210A, D350Y

xlix) Y67F, D350Y

l) A159V, D350Y

li) Y67F, D99G, A159V, V213I, L243P, L335M

lii) E109A, A159V, L314P, V320E, V399A, V429D

liii) A159V, L335M

liv) Y67F, A159V, L335M, D350Y

lv) D38N, L131P, A159V

lvi) T91S, L131P, A159V, A294T, R365H, L396M, D350Y

lvii) R183H, P210A, D350Y

lviii) Y67F, A159V, D350Y

lix) A159V, P210A, A294T, D350N

lx) Y67F, A159V, D350N

lxi) A159V, L335M, D350Y

lxii) P210A, A294T, D350Y

lxiii) T91S, A159V, A294T

lxiv) P210A, A294T, L335M, or

lxv) Y67F, L335M.

More preferably, the polypeptide comprises an amino acid sequence as provided in SEQ ID NO:1 with one of the following amino acid substitutions or groups of substitutions, or a substitutions at an amino acid position(s) corresponding thereto;

liv) Y67F, A159V, L335M, D350Y

lv) D38N, L131P, A159V

lvi) T91S, L131P, A159V, A294T, R365H, L396M, D350Y

lvii) R183H, P210A, D350Y

lviii) Y67F, A159V, D350Y

lix) A159V, P210A, A294T, D350N

lx) Y67F, A159V, D350N

lxi) A159V, L335M, D350Y

lxii) P210A, A294T, D350Y

lxiii) T91S, A159V, A294T

lxiv) P210A, A294T, L335M, or

lxv) Y67F, L335M.

In a particularly preferred embodiment, the polypeptide comprises a threonine or valine at a position corresponding to amino acid number 159 of SEQ ID NO:1, and when expressed in a bacterial cell more of the polypeptide is produced in soluble biologically active form than by an isogenic bacterial cell cultured under identical conditions comprising an exogenous polynucleotide comprising a nucleotide sequence provided as SEQ ID NO:2 or SEQ ID NO:4. Furthermore, in this embodiment it is preferred that the polypeptide additionally comprises i) an asparagine at a position corresponding to amino acid number 38 of SEQ ID NO:1, and ii) a proline, asparagine, threonine, aspartic acid, valine, glycine, cysteine, serine, glutamine, histidine, tyrosine or isoleucine at a position corresponding to amino acid number 131 of SEQ ID NO:1.

Preferably, if not specified otherwise, at a given amino acid position the polypeptide comprises an amino acid as found at the corresponding position of the polypeptide provided as SEQ ID NO:1.

If an amino acid at a nominated site is inconsistent with an amino acid substitution provided in Table 1, the nominated amino acid is preferred.

In a preferred embodiment, the polypeptide is a dimer of two separate polypeptide chains of the invention. The polypeptide may be a homodimer or a heterodimer. With regard to the heterodimer, it is preferred that the two polypeptide chains are at least 90%, more preferably at least 95%, more preferably at least 97%, more preferably at least 99%, identical.

In a further preferred embodiment, the polypeptide is associated with Zn² or Co²⁺.

In addition, when designing further mutants the skilled person can use the information provided in Example 2 regarding the structure of TrzNcc3.2.

Furthermore, if desired, unnatural amino acids or chemical amino acid analogues can be introduced as a substitution or addition into a polypeptide described herein. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide.

Polypeptides described herein can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

In an embodiment, a polypeptide of the invention comprises a signal sequence which is capable of directing secretion of the polypeptide from a cell. A large number of such signal sequences have been isolated, which include N- and C-terminal signal sequences. Prokaryotic and eukaryotic N-terminal signal sequences are similar, and it has been shown that eukaryotic N-terminal signal sequences are capable of functioning as secretion sequences in bacteria. An example of such an N-terminal signal sequence is the bacterial β-lactamase signal sequence, which is a well-studied sequence, and has been widely used to facilitate the secretion of polypeptides into the external environment. An example of C-terminal signal sequences is the hemolysin A (hlyA) signal sequences of E. coli. Additional examples of signal sequences include, without limitation, aerolysin, alkaline phosphatase gene (phoA), chitinase, endochitinase, α-hemolysin, MIpB, pullulanase, Yops and a TAT signal peptide.

### Polynucleotides

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 50%, more preferably at least 60%, more preferably at least 70%, more preferably at least 75%, more preferably at least 80%, at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

The present invention also relates to a polynucleotide which hybridizes under stringent conditions to a polynucleotide encoding SEQ ID NO:2 and/or SEQ ID NO:4 and which encodes a polypeptide of the invention and/or comprises a substitution(s) as defined herein. The term “stringent hybridization conditions” or “stringent conditions” and the like as used herein refers to parameters with which the art is familiar, including the variation of the hybridization temperature with length of an polynucleotide or oligonucleotide. Nucleic acid hybridization parameters may be found in references which compile such methods, Sambrook, et al., (supra), and Ausubel, et al., (supra). For example, stringent hybridization conditions, as used herein, can refer to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA) and washing twice in 0.2×SSC, 0.1% SDS at 65° C., with each wash step being about 30 min.

In a particularly preferred embodiment, the polynucleotide comprises a cytosine at a position corresponding to nucleotide number 468 of SEQ ID NO:2 or SEQ ID NO:4, and when expressed in a bacterial cell more of the polypeptide is produced than by an isogenic bacterial cell cultured under identical conditions comprising an exogenous polynucleotide comprising a nucleotide sequence provided as SEQ ID NO:2 or SEQ ID NO:4.

In a further particularly preferred embodiment, the polynucleotide encodes a polypeptide which comprises a threonine or valine at a position corresponding to amino acid number 159 of SEQ ID NO:1, and when expressed in a bacterial cell more of the polypeptide is produced in soluble biologically active form than by an isogenic bacterial cell cultured under identical conditions comprising an exogenous polynucleotide comprising a nucleotide sequence provided as SEQ ID NO:2 or SEQ ID NO:4. Furthermore, in this embodiment it is preferred that the polynucleotide additionally encodes a polypeptide which comprises i) an asparagine at a position corresponding to amino acid number 38 of SEQ ID NO:1, and ii) a proline, asparagine, threonine, aspartic acid, valine, glycine, cysteine, serine, glutamine, histidine, tyrosine or isoleucine at a position corresponding to amino acid number 131 of SEQ ID NO:1.

Polynucleotides of the present invention may possess, when compared to molecules provided herewith, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide are linked by phosphodiester bonds or analogs thereof. Analogs of phosphodiester linkages include: phosphorothioate, phosphorodithioate, phosphoroselenoate, phosphorodiselenoate, phosphoroanilothioate, phosphoranilidate and phosphoramidate.

### Recombinant Vectors

One embodiment of the present invention includes a recombinant vector, which comprises at least one isolated/exogenous polynucleotide of the invention inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or animal cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

### Host Cells

Another embodiment of the present invention includes a host cell transformed with one or more recombinant molecules described herein or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides described herein or can be capable of producing such polypeptides after being transformed with at least one polynucleotide molecule as described herein. Host cells of the present invention can be any cell capable of producing at least one protein defined herein, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Useful yeast cells include Pichia sp., Aspergillus sp. and Saccharomyces sp. Particularly preferred host cells are bacterial cells, yeast cells or plant cells.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

Plants contemplated for use in the practice of the present invention include both monocotyledons and dicotyledons. Target plants include, but are not limited to, the following: cereals (for example, wheat, barley, rye, oats, rice, maize, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (peanut, rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as tobacco, nuts, coffee, sugar cane, tea, vines, hops, turf, bananas and natural rubber plants, as well as ornamentals (flowers, shrubs, broad-leaved trees and evergreens, such as conifers). Crops frequently effected by Aspergillus sp. infection which are target plants of the invention include, but are not limited to, cereals (maize, sorghum, pearl millet, rice, wheat), oilseeds (peanut, soybean, sunflower, cotton), spices (chile peppers, black pepper, coriander, turmeric, ginger), and tree nuts (almond, pistachio, walnut, coconut). In a preferred embodiment, the plant is selected from sugar, cotton, corn, sorghum, pineapple, conifers such as christmas trees, eucalypts, wheat, oats, barley, rice and canola.

The term “plant” as used herein as a noun refers to a whole plants such as, for example, a plant growing in a field for commercial wheat production. A “plant part” refers to vegetative structures (for example, leaves, stems), roots, floral organs/structures, seed (including embryo, endosperm, and seed coat), plant tissue (for example, vascular tissue, ground tissue, and the like), cells and progeny of the same.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A “transgenic plant” refers to a plant that contains a gene construct (“transgene”) not found in a wild-type plant of the same species, variety or cultivar. A “transgene” as referred to herein has the normal meaning in the art of biotechnology and includes a genetic sequence which has been produced or altered by recombinant DNA or RNA technology and which has been introduced into the plant cell. The transgene may include genetic sequences derived from a plant cell. Typically, the transgene has been introduced into the plant by human manipulation such as, for example, by transformation but any method can be used as one of skill in the art recognizes

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α,β binding protein gene promoter. These promoters have been used to create DNA vectors that have been expressed in plants; see, e.g., WO 84/02913. All of these promoters have been used to create various types of plant-expressible recombinant DNA vectors.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H³⁰ symporter promoter, and the promoter for the thylakoid membrane protein genes from spinach (PsaD, PsaF, PsaE, PC, FNR, AtpC, AtpD, Cab, RbcS).

Other promoters for the chlorophyll α,β-binding proteins may also be utilized in the present invention, such as the promoters for LhcB gene and PsbP gene from white mustard (Sinapis alba). A variety of plant gene promoters that are regulated in response to environmental, hormonal, chemical, and/or developmental signals, also can be used for expression of RNA-binding protein genes in plant cells, including promoters regulated by (1) heat, (2) light (e.g., pea RbcS-3A promoter, maize RbcS promoter); (3) hormones, such as abscisic acid, (4) wounding (e.g., WunI); or (5) chemicals, such as methyl jasminate, salicylic acid, steroid hormones, alcohol, Safeners (WO 97/06269), or it may also be advantageous to employ (6) organ-specific promoters.

For the purpose of expression in sink tissues of the plant, such as the tuber of the potato plant, the fruit of tomato, or the seed of soybean, canola, cotton, Zea mays, wheat, rice, and barley, it is preferred that the promoters utilized in the present invention have relatively high expression in these specific tissues. A number of promoters for genes with tuber-specific or -enhanced expression are known, including the class I patatin promoter, the promoter for the potato tuber ADPGPP genes, both the large and small subunits, the sucrose synthase promoter, the promoter for the major tuber proteins including the 22 kD protein complexes and proteinase inhibitors, the promoter for the granule bound starch synthase gene (GBSS), and other class I and II patatins promoters. Other promoters can also be used to express a protein in specific tissues, such as seeds or fruits. The promoter for β-conglycinin or other seed-specific promoters such as the napin and phaseolin promoters, can be used. A particularly preferred promoter for Zea mays endosperm expression is the promoter for the glutelin gene from rice, more particularly the Osgt-1 promoter. Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins, and the glutenins Examples of such promoters in rice include those promoters for the ADPGPP subunits, the granule bound and other starch synthase, the branching enzymes, the debranching enzymes, sucrose synthases, and the glutelins. A particularly preferred promoter is the promoter for rice glutelin, Osgt-1 gene. Examples of such promoters for barley include those for the ADPGPP subunits, the granule bound and other starch synthase, the branching enzymes, the debranching enzymes, sucrose synthases, the hordeins, the embryo globulins, and the aleurone specific proteins.

Root specific promoters may also be used. An example of such a promoter is the promoter for the acid chitinase gene. Expression in root tissue could also be accomplished by utilizing the root specific subdomains of the CaMV 35S promoter that have been identified.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). The 5′ non-translated regions can also be obtained from plant viral RNAs (Tobacco mosaic virus, Tobacco etch virus, Maize dwarf mosaic virus, Alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. No. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) are coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. An illustrative embodiment of a method for delivering DNA into Zea mays cells by acceleration is a biolistics α-particle delivery system, that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun, available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus that express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. No. 5,451,513, U.S. Pat. No. 5,545,818, U.S. Pat. No. 5,877,402, U.S. Pat. No. 5,932,479, and WO 99/05265).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described (Klee et al., In: Plant DNA Infectious Agents, Hohn and Schell, eds., Springer-Verlag, New York, pp. 179-203 (1985)). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, In: Breeding Methods for Cultivar Development, Wilcox J. ed., American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., In: Methods for Plant Molecular Biology, Academic Press, San Diego, Calif., (1988)). This regeneration and growth process typically includes the steps of selection of transformed cells; culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. No. 5,589,617, U.S. Pat. No. 6,541,257, and WO 99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

In an embodiment, transgenic plants of the invention are produced using methods generally described in U.S. Pat. No. 6,369,299.

### Transgenic Non-Human Animals

A “transgenic non-human animal” refers to an animal, other than a human, that contains a gene construct (“transgene”) not found in a wild-type animal of the same species or breed. A “transgene” as referred to herein has the normal meaning in the art of biotechnology and includes a genetic sequence which has been produced or altered by recombinant DNA or RNA technology and which has been introduced into an animal cell. The transgene may include genetic sequences derived from an animal cell. Typically, the transgene has been introduced into the animal by human manipulation such as, for example, by transformation but any method can be used as one of skill in the art recognizes

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention include excipients, also referred to herein as “acceptable carriers”. An excipient can be any material that the animal, plant, plant or animal material, or environment (including soil and water samples) to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

In an embodiment, a polypeptide of the invention is immobilized on a solid support. This can enhance the rate and/or degree of hydrolysis of an s-triazine or diazine, and/or increase the stability of the polypeptide. For example, the polypeptide can be immobilized on a polyurethane matrix (Gordon et al., 1999), or encapsulated in appropriate liposomes (Petrikovics et al., 2000a and b). The polypeptide can also be incorporated into a composition comprising a foam such as those used routinely in fire-fighting (LeJeune et al., 1998). As would be appreciated by the skilled addressee, the polypeptide of the present invention could readily be used in a sponge or foam as disclosed in WO 00/64539. Other solid supports useful for the invention include resins with an acrylic type structure, with epoxy functional groups, such as Sepabeads EC-EP (Resindion srl—Mitsubishi Chemical Corporation) and Eupergit C (Rohm-Degussa), or with primary amino groups, such as Sepabeads EC-has and EC-EA (Resindion srl—Mitsubishi Chemical Corporation). In any case, the polypeptide is brought in contact with the resin and immobilized through the high reactivity of the functional groups (epoxides) or activation of the resin with a bifunctional agent, such as glutaraldehyde, so as to bind the enzyme to the matrix. Other resins suitable for the invention are polystyrene resins, macroreticular resins and resins with basic functional groups, such as Sepabeads EC-Q1A: the polypeptide is absorbed on the resin and then stabilized by cross-linking with a bifunctional agent (glutaraldehyde).

In an embodiment, the composition comprises Zn²⁺ and/or Co²⁺. In another embodiment, a method of the invention for hydrolysing an s-triazine or diazine comprises providing Zn²⁺ and/or Co²⁺ as a co-factor for a polypeptide of the invention.

One embodiment of the present invention is a controlled release formulation that is capable of slowly releasing a composition of the present invention into an animal, plant, animal or plant material, or the environment (including soil and water samples). As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

A preferred controlled release formulation of the present invention is capable of releasing a composition of the present invention into soil or water which is in an area comprising a s-triazine or diazine. The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

The concentration of the polypeptide, vector, or host cell etc of the present invention that will be required to produce effective compositions for hydrolysing an s-triazine or diazine, will depend on the nature of the sample to be decontaminated, the concentration of the s-triazine or diazine in the sample, and the formulation of the composition. The effective concentration of the polypeptide, vector, or host cell etc within the composition can readily be determined experimentally using a method of the invention.

Enzymes of the invention, and/or host cells encoding therefor, can be used in coating compositions as generally described in WO 2004/112482 and WO 2005/26269.

### EXAMPLES

### Example 1

### TrzN Mutants with Enhanced Activity

### Methods and Materials

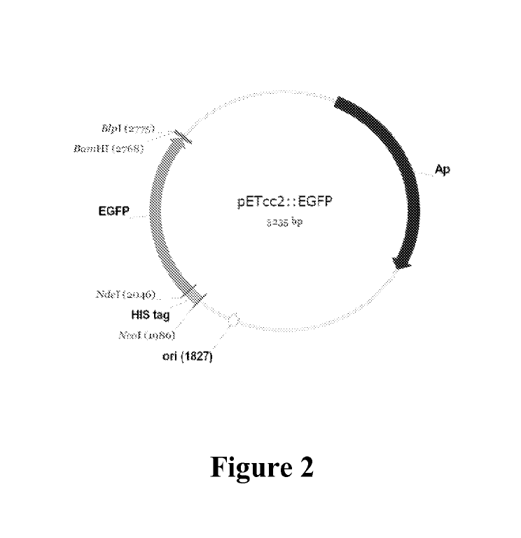

A truncated pET14b plasmid (pETcc2) was used for the expression of TrzN and its variants. All trzN genes were cloned into pETcc2 using the unique NdeI and BamHI sites, and expressed in E. coli strain BL21 λDE3 (Novagen). NdeI/BamHI digested pETcc2 was prepared from pETcc2::egfp (FIG. 2), which provided a simple visual indication of the proportion of religated vector in the libraries (i.e. relegated vector fluoresced strongly under blue light).

Codon optimised TrzN (SEQ ID NO:2—TrzNco) was produced by GENEART AG (BioPark, Josef-Engert-Str. 11, D-93053 Regensburg Germany).

Random mutagenesis was performed using GeneMorph II (Stratagen) according to the manufactures instructions. Oligonucleotide primers 1 and 2 were used to amplify the gene (Table 2).

Site-saturation mutagenesis was performed by PCR mediated site-directed mutagenesis using primers 3-24, as detailed in Table 2. The NNS degeneracy (Georgescu et al., in Directed Evolution Library Creation, Eds.: F. H. Arnold, G. Georgiou, Humana Press, Totowa, N.J., 2003, pp. 75-89) was used to generate diversity at the 67^{th}, 91^{st}, 131^{st}, 159^{th}, 161^{st}, 243^{rd}, 246^{th}, 294^{th}, 335^{th}, and 350^{th} codons of the trzN gene (where N is any nucleotide and S is either G or C).

Random and site-saturated libraries were screened using LB agar plates supplemented with 200 μg.mL⁻¹ ampicillin, 1 μM IPTG and impregnated with 1 mg.mL⁻¹ atrazine (90% atrazine w/w; Gesaprim 900 WG, Syngenta). TrzN activity was assessed by atrazine dechlorination, which resulted in clarification of the medium in the vicinity of colonies expressing active TrzN. The level of activity was determined by the rate at which the clarification occurred.

All sequencing was performed by Micromon (Monash University, Melbourne, Victoria).

Substrate range was tested by measuring the rate of substrate hydrolysis by purified His₆TrzNcc3.2, as measured by the loss of absorbance at 264 nM as previously reported (Shapir et al., 2005b). Atrazine, ametryn, propazine, prometryn, simazine, simetryn, ipazine, trietazine and cyanozine (>99% purity Pestinal standards; Sigma) were tested by this method. His₆TrzNcc3.2 was purified by affinity chromatography (H isTrap; GE HealthCare), followed by size exclusion chromatography (Superdex 200; GE HealthCare).

TABLE 2
Oligonucleotide primers used in this study.

| No | Application | Sequence (5′-3′) |
|---|---|---|
| 1 | trzNco forward | CCACAACATATGATTCTGATCCGTGGTCTGACCCGCGTTATC |
| | | (SEQ ID NO: 5) |
| 2 | trzNco reverse | CTTCGAATTCTTACAGATTTTTCGGAATCAGGGCCGTGGTATTAG |
| | | (SEQ ID NO: 6) |
| 3 | 67X mutagenesis forward | CGCACGATCGATGGTCGCGGCATGATTGCCCTGCCGGGTCTGATCAAT |
| | | AGCCACCAGCATCTGNNSGAGGGC (SEQ ID NO: 7) |
| 4 | 67X mutagenesis reverse | CAGATGCTGGTGGCTATTGATCAGACCCGGCAGGGCAATCATGCCGCG |
| | | ACCATCGATCGTGC (SEQ ID NO: 8) |
| 5 | 91X mutagenesis forward | GCGAGCTGGTTAGAGGGCGTCCTGNNSCGTAGCGCGGGTTGGTGGCGT |
| | | (SEQ ID NO: 9) |
| 6 | 91X mutagenesis reverse | ACGCCACCAACCCGCGCTACG (SEQ ID NO: 10) |
| 7 | 131X mutagenesis forward | TCCTGCTGGAAAGCCTGCTGGGCGGTATCACCACCGTCGCCGATCAGC |
| | | ATNNSTTTTTTCCAGGTGCAACCGC (SEQ ID NO: 11) |
| 8 | 131X mutagenesis reverse | ATGCTGATCGGCGACGGTGGTGATACCGCCCAGCAGGCTTTCCAGCAGGA |
| | | (SEQ ID NO: 12) |
| 9 | 159X mutagenesis forward | TATATTGATGCAACCATCGAAGCCGCGACCGACCTGGGTATTCGCTTTCA |
| | | TGCCNNSCGCAGCAGCATG (SEQ ID NO: 13) |
| 10 | 159X mutagenesis reverse | GGCATGAAAGCGAATACCCAGGTCGGTCGCGGCTTCGATGGTTGCATCAAT |
| | | ATA (SEQ ID NO: 14) |
| 11 | 161X mutagenesis forward | ACCATCGAAGCCGCGACCGACCTGGGTATTCGCTTTCATGCCGCGCGCNNS |
| | | AGCATGACTCTGGGTAAG (SEQ ID NO: 15) |
| 12 | 161X mutagenesis reverse | GCGCGCGGCATGAAAGCGAATACCCAGGTCGGTCGCGGCTTCGATGGT |
| | | (SEQ ID NO: 16) |
| 13 | 210X mutagenesis forward | GAACCATTTGGCATGGTCCGCATTGCACTGGGTNNSTGCGGCGTTCCGTAT |
| | | (SEQ ID NO: 17) |
| 14 | 210X mutagenesis reverse | ACCCAGTGCAATGCGGACCATGCCAAATGGTTC (SEQ ID NO: 18) |
| 15 | 243X mutagenesis forward | GTGCGCCTGCATACGCATTTTTATGAACCGNNSGACGCG (SEQ ID NO: 19) |
| 16 | 243X mutagenesis reverse | CGGTTCATAAAAATGCGTATGCAGGCGCAC (SEQ ID NO: 20) |
| 17 | 246X mutagenesis forward | CTGCATACGCATTTTTATGAACCGCTGGACGCGNNSATGAGC |
| | | (SEQ ID NO: 21) |
| 18 | 246X mutagenesis reverse | CGCGTCCAGCGGTTCATAAAAATGCGTATGCAG (SEQ ID NO: 22) |
| 19 | 294X mutagenesis forward | ATTCCAGAATTTGCGGATGCCGGCGTTGCAATTNNSCACCTGATTGCGCCG |
| | | GATCTGCGTC (SEQ ID NO: 23) |
| 20 | 294X mutagenesis reverse | AATTGCAACGCCGGCATCCGCAAATTCTGGAAT (SEQ ID NO: 24) |
| 21 | 335X mutagenesis forward | ACCGGTAGCGCCAGCAACGACGGTGGCAACNNSTTAGGTGATCTG |
| | | (SEQ ID NO: 25) |
| 22 | 335X mutagenesis reverse | GTTGCCACCGTCGTTGCTGGCGCTACCGGT (SEQ ID NO: 26) |
| 23 | 350X mutagenesis forward | TTAGGTGATCTGCGCCTGGCAGCCCTGGCGCATCGCCCGGCGNNSCCGAATGAA |
| | | (SEQ ID NO: 27) |
| 24 | 350X mutagenesis reverse | CGCCGGGCGATGCGCCAGGGCTGCCAGGCGCAGATCACCTAA |
| | | (SEQ ID NO: 28) |
| 25 | 38X mutagenesis forward | GCTGTGGGTAAANNSTTAAGCGATCGTAG (SEQ ID NO: 29) |
| 26 | 38X mutagenesis reverse | CTACGATCGCTTAASNNTTTACCCACAGC (SEQ ID NO: 30) |

### Results

### Iterative Random Mutagenesis

An atrazine plate-clearing assay was used to assess the ability of BL21 λDE3 pETcc2::trzNco to dechlorinate atrazine hydrolytically. As no clarification of the solid medium in the vicinity of the bacterial growth was observed after 30 days, BL21 λDE3 pETcc2::trzNco was deemed to have no, or undetectable, levels of atrazine chlorohydrolase activity. Random mutants of trzNco were generated using the low fidelity DNA polymerase Mutazyme II (GeneMorph II), and screened on atrazine containing agar plates for hydrolytic activity against atrazine. A single synonymous mutation in trzNco (T468C) altered the phenylalanine encoding 156^{th} codon from TTT to TTC (SEQ ID NO:3). The mutant (TrzN L1, Table 3) conferred the ability to clear atrazine after eight days at 37° C.

TrzN L1 was used as template for the next round of random mutagenesis (iteration 1). Twenty-seven mutants were found that conferred upon BL21 the ability to form zones of clearance more rapidly than the parent trzNco parent (TrzN L1, Table 3). Zones of clearance appeared at between three and six days for the iteration 1 mutants. The fifteen (TrzN cc1.1, TrzN cc1.3, TrzN cc1.4, TrzN cc1.6, TrzN cc1.8, TrzN cc1.9, TrzN cc1.10, TrzN cc1.11, TrzN cc1.12, TrzN cc1.13, TrzN cc1.14, TrzN cc1.15, TrzN cc1.25, TrzN cc1.26 and TrzN cc1.27) mutants with the most rapidly forming zones of clearance were used as templates for another round of random mutagenesis (iteration 2).

The products from the iteration 2 mutagenic PCR were screened together, so that the best overall performing mutants would be selected. Thirty six colonies were selected that formed zones of clearance from 45-96 hours (Table 3). The best thirteen iteration 2 mutants (clearing between 45-48 hours; TrzN cc2.1, TrzN cc2.2, TrzN cc2.3, TrzN cc2.4, TrzN cc2.5, TrzN cc2.6, TrzN cc2.7, TrzN cc2.8, TrzN cc2.9, TrzN cc2.10, TrzN cc2.11, TrzN cc2.12 and TrzN cc2.13) were used as templates for the third iteration of random mutagenesis.

TABLE 3
Mutants generated by iterative random mutagenesis. Rate of
clearing judged on 1% atrazine (w/v), 1 mM IPTG containing
LB agar plates incubated at 37° C. The mutants used as
template in the subsequent iteration of random mutagenesis
are indicated.

| Mutant ID | Time to clear | Comments |
|---|---|---|
| AtzA | 2 days | Alternative atrazine chlorohydrolase |
| TrzN | None | Codon optimised wildtype |
| Wildtype | | |
| TrzN L1 | 8 days | Iteration 0 mutant - template for Iteration 1 |
| TrzN cc1.1 | 4 days | Iteration 1 mutant - template for Iteration 2 |
| TrzN cc1.3 | 3 days | Iteration 1 mutant - template for Iteration 2 |
| TrzN cc1.4 | 3 days | Iteration 1 mutant - template for Iteration 2 |
| TrzN cc1.6 | 3 days | Iteration 1 mutant - template for Iteration 2 |
| TrzN cc1.8 | 3 days | Iteration 1 mutant - template for Iteration 2 |
| TrzN cc1.9 | 4 days | Iteration 1 mutant - template for Iteration 2 |
| TrzN cc1.10 | 3 days | Iteration 1 mutant - template for Iteration 2 |
| TrzN cc1.11 | 4 days | Iteration 1 mutant - template for Iteration 2 |
| TrzN cc1.12 | 4 days | Iteration 1 mutant - template for Iteration 2 |
| TrzN cc1.13 | 4 days | Iteration 1 mutant - template for Iteration 2 |
| TrzN cc1.14 | 4 days | Iteration 1 mutant - template for Iteration 2 |
| TrzN cc1.15 | 4 days | Iteration 1 mutant - template for Iteration 2 |
| TrzN cc1.17 | 5 days | Iteration 1 mutant |
| TrzN cc1.25 | 4 days | Iteration 1 mutant - template for Iteration 2 |
| TrzN cc1.26 | 3 days | Iteration 1 mutant - template for Iteration 2 |
| TrzN cc1.27 | 4 days | Iteration 1 mutant - template for Iteration 2 |
| TrzN cc1.30 | 6 days | Iteration 1 mutant |
| TrzN cc1.31 | 6 days | Iteration 1 mutant |
| TrzN cc1.36 | 6 days | Iteration 1 mutant |
| TrzN cc1.40 | 6 days | Iteration 1 mutant |
| TrzN cc1.43 | 6 days | Iteration 1 mutant |
| TrzN cc1.44 | 6 days | Iteration 1 mutant |
| TrzN cc1.47 | 6 days | Iteration 1 mutant |
| TrzN cc1.49 | 6 days | Iteration 1 mutant |
| TrzN cc1.53 | 6 days | Iteration 1 mutant |
| TrzN cc1.58 | 6 days | Iteration 1 mutant |
| TrzN cc1.65 | 6 days | Iteration 1 mutant |
| TrzN cc2.1 | 45 hr | Iteration 2 mutant - template for Iteration 3 |
| TrzN cc2.2 | 45 hr | Iteration 2 mutant - template for Iteration 3 |
| TrzN cc2.3 | 45 hr | Iteration 2 mutant - template for Iteration 3 |
| TrzN cc2.4 | 45 hr | Iteration 2 mutant - template for Iteration 3 |
| TrzN cc2.5 | 45 hr | Iteration 2 mutant - template for Iteration 3 |
| TrzN cc2.6 | 45 hr | Iteration 2 mutant - template for Iteration 3 |
| TrzN cc2.7 | 48 hr | Iteration 2 mutant - template for Iteration 3 |
| TrzN cc2.8 | 48 hr | Iteration 2 mutant - template for Iteration 3 |
| TrzN cc2.9 | 48 hr | Iteration 2 mutant - template for Iteration 3 |
| TrzN cc2.10 | 48 hr | Iteration 2 mutant - template for Iteration 3 |
| TrzN cc2.11 | 48 hr | Iteration 2 mutant - template for Iteration 3 |
| TrzN cc2.12 | 48 hr | Iteration 2 mutant - template for Iteration 3 |
| TrzN cc2.13 | 48 hr | Iteration 2 mutant - template for Iteration 3 |
| TrzN cc2.15 | 72 hr | Iteration 2 mutant |
| TrzN cc2.16 | 48 hr | Iteration 2 mutant |
| TrzN cc2.17 | 48 hr | Iteration 2 mutant |
| TrzN cc2.18 | 48 hr | Iteration 2 mutant |
| TrzN cc2.19 | 48 hr | Iteration 2 mutant |
| TrzN cc2.20 | 48 hr | Iteration 2 mutant |
| TrzN cc2.21 | 48 hr | Iteration 2 mutant |
| TrzN cc2.22 | 48 hr | Iteration 2 mutant |
| TrzN cc2.23 | 48 hr | Iteration 2 mutant |
| TrzN cc2.24 | 48 hr | Iteration 2 mutant |
| TrzN cc2.25 | 48 hr | Iteration 2 mutant |
| TrzN cc2.26 | 48 hr | Iteration 2 mutant |
| TrzN cc2.27 | 48 hr | Iteration 2 mutant |
| TrzN cc2.28 | 48 hr | Iteration 2 mutant |
| TrzN cc2.29 | 48 hr | Iteration 2 mutant |
| TrzN cc2.30 | 48 hr | Iteration 2 mutant |
| TrzN cc2.31 | 48 hr | Iteration 2 mutant |
| TrzN cc2.32 | 48 hr | Iteration 2 mutant |
| TrzN cc2.33 | 48 hr | Iteration 2 mutant |
| TrzN cc2.34 | 48 hr | Iteration 2 mutant |
| TrzN cc2.35 | 48 hr | Iteration 2 mutant |
| TrzN cc2.36 | 48 hr | Iteration 2 mutant |
| TrzN cc2.37 | 96 hr | Iteration 2 mutant |
| TrzN cc3.1 | 24 hr | Iteration 3 mutant |
| TrzN cc3.2 | 26 hr | Iteration 3 mutant |
| TrzN cc3.3 | 26 hr | Iteration 3 mutant |
| TrzN cc3.4 | 26 hr | Iteration 3 mutant |
| TrzN cc3.5 | 26 hr | Iteration 3 mutant |
| TrzN cc3.6 | 26 hr | Iteration 3 mutant |
| TrzN cc3.7 | 26 hr | Iteration 3 mutant |
| TrzN cc3.8 | 31 hr | Iteration 3 mutant |
| TrzN cc3.9 | 31 hr | Iteration 3 mutant |
| TrzN cc3.10 | 31 hr | Iteration 3 mutant |
| TrzN cc3.11 | 31 hr | Iteration 3 mutant |
| TrzN cc3.12 | 31 hr | Iteration 3 mutant |
| TrzN cc3.13 | 31 hr | Iteration 3 mutant |

Again, the products from the iteration 2 mutagenic PCR were screened together, so that the best overall performing mutants would be selected. Thirteen third iteration mutants were selected; conferring clearing times of 24-31 hours were selected. The nucleotide and amino acid replacements in the mutants are summarized in Tables 4 and 5 respectively. The largest number of nucleotide mutations within a single gene was fourteen, with up to seven amino acid substitutions in any variant enzyme.

TABLE 4
Summary of the mutations in the improved trzN genes.

| Mutant | | Nucleotide substitutions | | | | | |
|---|---|---|---|---|---|---|---|
| TrzNL1 | T468C | | | | | | |
| TrzNcc1.1 | T468C | A938T | | | | | |
| TrzNcc1.3 | A200T | G210A | T468C | | | | |
| TrzNcc1.4 | T468C | C476T | G753C | | | | |
| TrzNcc1.6 | T468C | C476T | T728C | | | | |
| TrzNcc1.8 | T468C | G1048T | | | | | |
| TrzNcc1.9 | T384C | T468C | G569A | G681T | | | |
| TrzNcc1.10 | T468C | G475A | | | | | |
| TrzNcc1.11 | C279T | T468C | C1223T | C1329T | | | |
| TrzNcc1.12 | C76A | T468C | A481G | | | | |
| TrzNcc1.13 | C39A | C101A | T468C | T639C | G737C | G1048T | |
| TrzNcc1.14 | C410T | A418C | T468C | A600G | | | |
| TrzNcc1.15 | T468C | G705A | C1003A | | | | |
| TrzNcc1.17 | T468C | G573C | | | | | |
| TrzNcc1.25 | T468C | C474T | C628G | C1278T | | | |
| TrzNcc1.26 | T399C | T468C | G880A | T900C | | | |
| TrzNcc1.27 | T468C | C1236T | | | | | |
| TrzNcc1.30 | T87C | A367G | T468C | | | | |
| TrzNcc1.31 | T468C | G1176T | | | | | |
| TrzNcc1.36 | C135T | T468C | C1344A | | | | |
| TrzNcc1.40 | T468C | A852T | | | | | |
| TrzNcc1.43 | T468C | T738C | | | | | |
| TrzNcc1.44 | C454T | T468C | | | | | |
| TrzNcc1.47 | A200T | T468C | G1309A | | | | |
| TrzNcc1.49 | T468C | G489T | G745A | | | | |
| TrzNcc1.53 | C410T | T468C | | | | | |
| TrzNcc1.58 | T468C | G736A | | | | | |
| TrzNcc1.65 | G225T | C268A | A414G | T468C | T627C | C1321T | |
| TrzNcc2.1 | A432T | T468C | T471C | C476T | T728C | G1053A | C1351A |
| TrzNcc2.2 | A303G | C449G | T468C | C476T | C686T | C690A | T728C |
| TrzNcc2.3 | A200T | G210A | C372T | T468C | A654G | C1003A | |
| TrzNcc2.4 | C180T | A200T | A302G | C375A | T399C | C411T | T468C |
| TrzNcc2.5 | C229T | T468C | G705A | C1003A | | | |
| TrzNcc2.6 | T317C | T468C | A481G | T531A | G753C | T906A | T978A |
| TrzNcc2.7 | A127G | T320C | T468C | C476T | G753C | G1048T | |
| TrzNcc2.8 | A157T | C410T | A418C | T468C | A545G | G568A | A600G |
| TrzNcc2.9 | T468C | C476T | A723G | C1003A | G1048T | C1329T | |
| TrzNcc2.10 | C84A | T399C | T468C | C483T | G880A | T900C | G1048A |
| TrzNcc2.11 | T468C | T498C | C628G | C885T | A1086G | G1270A | |
| TrzNcc2.12 | T384C | T468C | C476T | G569A | G753C | A1152G | |
| TrzNcc2.13 | T336C | T468C | C476T | A537G | C564T | | |
| TrzNcc2.15 | A228G | T240C | T468C | C628G | G880A | G1048T | G1094A |
| TrzNcc2.16 | T273A | A367G | C381T | T399C | T468C | A481G | G880A |
| TrzNcc2.17 | A200T | C207T | G210A | T468C | C476T | G1048T | A1059G |
| TrzNcc2.18 | A271T | A333G | T399C | T468C | C476T | A843G | G880A |
| TrzNcc2.19 | A200T | G210A | C346T | T468C | C476T | T579C | T728C |
| TrzNcc2.20 | C438T | T468C | C476T | A855G | | | |
| TrzNcc2.21 | G168A | T426A | T468C | C476T | C628G | C1203T | T1305C |
| TrzNcc2.22 | T468C | C476T | T663C | C1236T | | | |
| TrzNcc2.23 | T384C | T468C | C476T | T728C | A928G | C1003A | C1186A |
| TrzNcc2.24 | T5C | C315A | T468C | C476T | G589A | G681T | T728C |
| TrzNcc2.25 | C279T | T468C | C476T | C1003A | | | |
| TrzNcc2.26 | A250C | A314G | T468C | C476T | | | |
| TrzNcc2.27 | A200T | G210A | T468C | T555C | G880A | T900C | |
| TrzNcc2.28 | T468C | C476T | T546G | C696T | C1003A | G1048T | |
| TrzNcc2.29 | A200T | G210A | T468C | C476T | T728C | T1101G | |
| TrzNcc2.30 | G112A | C165T | T468C | C476T | T618C | G753C | C999T |
| TrzNcc2.31 | T423C | T468C | C476T | G489A | A584T | G753C | G1048T |
| TrzNcc2.32 | T466C | T468C | C474T | C628G | G1048T | | |
| TrzNcc2.33 | T108A | A200T | G210A | G357A | T468C | G1048T | A1059G |
| TrzNcc2.34 | T468C | C476T | G567A | G753C | G1048T | | |
| TrzNcc2.35 | A200T | G210A | A296G | T468C | C476T | G637A | T728C |
| TrzNcc2.36 | C189T | A326C | T468C | C476T | T941C | T959A | A1059G |
| TrzNcc2.37 | T468C | C476T | C1003A | | | | |
| TrzNcc3.1 | A200T | G210A | C279T | T468C | C476T | T879C | C1003A |
| TrzNcc3.2 | G112A | C165T | T392C | T468C | C476T | T618C | G660A |
| TrzNcc3.3 | A271T | T392C | T468C | C476T | G753C | G880A | G1048T |
| TrzNcc3.4 | A228G | T240C | T468C | G548A | C628G | G1048T | C1332T |
| TrzNcc3.5 | T108A | A200T | G210A | T423C | T468C | C476T | G567A |
| TrzNcc3.6 | T384C | T468C | C476T | C628G | C867T | G880A | T900C |
| TrzNcc3.7 | T384C | T468C | C476T | C628G | C867T | G880A | T900C |
| TrzNcc3.8 | A200T | C207T | G210A | T468C | C476T | T840A | T900C |
| TrzNcc3.9 | T468C | C476T | C633T | G660T | A723G | C1003A | G1048T |
| TrzNcc3.10 | A228G | T240C | G270A | T468C | C628G | G880A | G1048T |
| TrzNcc3.11 | A271T | A333G | T399C | T468C | C476T | A843G | G880A |
| TrzNcc3.12 | A228G | T240C | T468C | C628G | G880A | C957A | C1003A |
| TrzNcc3.13 | A200T | G210A | C411A | T468C | T555C | T639C | A654G |

| Mutant | Nucleotide substitutions | | | | | | |
|---|---|---|---|---|---|---|---|
| TrzNL1 | | | | | | | |
| TrzNcc1.1 | | | | | | | |
| TrzNcc1.3 | | | | | | | |
| TrzNcc1.4 | | | | | | | |
| TrzNcc1.6 | | | | | | | |
| TrzNcc1.8 | | | | | | | |
| TrzNcc1.9 | | | | | | | |
| TrzNcc1.10 | | | | | | | |
| TrzNcc1.11 | | | | | | | |
| TrzNcc1.12 | | | | | | | |
| TrzNcc1.13 | | | | | | | |
| TrzNcc1.14 | | | | | | | |
| TrzNcc1.15 | | | | | | | |
| TrzNcc1.17 | | | | | | | |
| TrzNcc1.25 | | | | | | | |
| TrzNcc1.26 | | | | | | | |
| TrzNcc1.27 | | | | | | | |
| TrzNcc1.30 | | | | | | | |
| TrzNcc1.31 | | | | | | | |
| TrzNcc1.36 | | | | | | | |
| TrzNcc1.40 | | | | | | | |
| TrzNcc1.43 | | | | | | | |
| TrzNcc1.44 | | | | | | | |
| TrzNcc1.47 | | | | | | | |
| TrzNcc1.49 | | | | | | | |
| TrzNcc1.53 | | | | | | | |
| TrzNcc1.58 | | | | | | | |
| TrzNcc1.65 | | | | | | | |
| TrzNcc2.1 | | | | | | | |
| TrzNcc2.2 | C1128T | | | | | | |
| TrzNcc2.3 | | | | | | | |
| TrzNcc2.4 | A540G | G880A | T900C | | | | |
| TrzNcc2.5 | | | | | | | |
| TrzNcc2.6 | C1003A | A1326G | | | | | |
| TrzNcc2.7 | | | | | | | |
| TrzNcc2.8 | C628G | G630A | G1048T | C1332T | | | |
| TrzNcc2.9 | | | | | | | |
| TrzNcc2.10 | | | | | | | |
| TrzNcc2.11 | | | | | | | |
| TrzNcc2.12 | | | | | | | |
| TrzNcc2.13 | | | | | | | |
| TrzNcc2.15 | | | | | | | |
| TrzNcc2.16 | T900C | | | | | | |
| TrzNcc2.17 | | | | | | | |
| TrzNcc2.18 | T900C | C1236T | | | | | |
| TrzNcc2.19 | C1278T | | | | | | |
| TrzNcc2.20 | | | | | | | |
| TrzNcc2.21 | | | | | | | |
| TrzNcc2.22 | | | | | | | |
| TrzNcc2.23 | | | | | | | |
| TrzNcc2.24 | G897A | C1003A | | | | | |
| TrzNcc2.25 | | | | | | | |
| TrzNcc2.26 | | | | | | | |
| TrzNcc2.27 | | | | | | | |
| TrzNcc2.28 | | | | | | | |
| TrzNcc2.29 | | | | | | | |
| TrzNcc2.30 | T1101C | | | | | | |
| TrzNcc2.31 | | | | | | | |
| TrzNcc2.32 | | | | | | | |
| TrzNcc2.33 | C1278T | | | | | | |
| TrzNcc2.34 | | | | | | | |
| TrzNcc2.35 | C1003A | | | | | | |
| TrzNcc2.36 | C1186T | T1196C | G1248T | T1286A | | | |
| TrzNcc2.37 | | | | | | | |
| TrzNcc3.1 | G1048T | | | | | | |
| TrzNcc3.2 | C675A | G753C | C807A | C993T | C999T | T1101C | T1305C |
| TrzNcc3.3 | G1094A | C1186A | G1221A | | | | |
| TrzNcc3.4 | | | | | | | |
| TrzNcc3.5 | G1048T | | | | | | |
| TrzNcc3.6 | C981T | G1048A | A1326G | | | | |
| TrzNcc3.7 | C981T | G1048A | A1326G | | | | |
| TrzNcc3.8 | G1048A | | | | | | |
| TrzNcc3.9 | C1329T | | | | | | |
| TrzNcc3.10 | | | | | | | |
| TrzNcc3.11 | T900C | C1003T | C1236T | | | | |
| TrzNcc3.12 | | | | | | | |
| TrzNcc3.13 | G768A | T774A | C972T | C1003A | T1011C | G1353T | |

TABLE 5
Summary of the mutations in the improved TrzN variant enzymes.

| Variant | Mutations | | | | | | |
|---|---|---|---|---|---|---|---|
| trzN_L1 | | | | | | | |
| TrzN cc1.1 | Y313F | | | | | | |
| TrzN_cc1.3 | Y67F | | | | | | |
| TrzN_cc1.4 | A159V | | | | | | |
| TrzN_cc1.6 | A159V | L243P | | | | | |
| TrzN_cc1.8 | D350Y | | | | | | |
| TrzN_cc1.9 | G190D | M227I | | | | | |
| TrzN_cc1.10 | A159T | | | | | | |
| TrzN_cc1.11 | A408V | | | | | | |
| TrzN_cc1.12 | L26M | S161G | | | | | |
| TrzN_cc1.13 | F13L | A34D | G246A | D350Y | | | |
| TrzN_cc1.14 | T137I | S140R | | | | | |
| TrzN_cc1.15 | L335M | | | | | | |
| TrzN_cc1.17 | | | | | | | |
| TrzN_cc1.25 | P210A | | | | | | |
| TrzN_cc1.26 | A294T | | | | | | |
| TrzN_cc1.27 | | | | | | | |
| TrzN_cc1.30 | I123V | | | | | | |
| TrzN_cc1.31 | | | | | | | |
| TrzN_cc1.36 | | | | | | | |
| TrzN_cc1.40 | | | | | | | |
| TrzN_cc1.43 | | | | | | | |
| TrzN_cc1.44 | | | | | | | |
| TrzN_cc1.47 | Y67F | V437I | | | | | |
| TrzN_cc1.49 | M163I | D249N | | | | | |
| TrzN_cc1.53 | T137I | | | | | | |
| TrzN_cc1.58 | G246S | | | | | | |
| TrzN_cc1.65 | L90M | | | | | | |
| TrzN_cc2.1 | L243P | A159V | L451M | | | | |
| TrzN_cc2.2 | T150S | A159V | A229V | D230E | L243P | | |
| TrzN_cc2.3 | Y67F | L335M | | | | | |
| TrzN_cc2.4 | Y67F | K101R | A294T | | | | |
| TrzN_cc2.5 | L335M | | | | | | |
| TrzN_cc2.6 | V106A | S161G | F177L | L335M | | | |
| TrzN_cc2.7 | S43G | I107T | A159V | D350Y | | | |
| TrzN_cc2.8 | M53L | T137I | S140R | D182G | G190S | D350Y | |
| TrzN_cc2.9 | A159V | L335M | D350Y | | | | |
| TrzN_cc2.10 | D28E | A294T | D350N | | | | |
| TrzN_cc2.11 | P210A | V424I | | | | | |
| TrzN_cc2.12 | A159V | G190D | | | | | |
| TrzN_cc2.13 | A159V | | | | | | |
| TrzN cc2.15 | P210A | A294T | R365H | D350Y | | | |
| TrzN cc2.16 | I123V | S161G | A294T | | | | |
| TrzN cc2.17 | Y67F | A159V | D350Y | | | | |
| TrzN cc2.18 | T91S | A159V | A294T | | | | |
| TrzN cc2.19 | Y67F | A159V | L243P | | | | |
| TrzN cc2.20 | A159V | | | | | | |
| TrzN cc2.21 | A159V | P210A | | | | | |
| TrzN cc2.22 | A159V | | | | | | |
| TrzN cc2.23 | A159V | L243P | I310V | L335M | L396M | | |
| TrzN cc2.24 | I2T | D105E | E197K | A159V | M227I | L243P | L335M |
| TrzN cc2.25 | A159V | L335M | | | | | |
| TrzN cc2.26 | S84R | D105G | A159V | | | | |
| TrzN cc2.27 | Y67F | A294T | | | | | |
| TrzN cc2.28 | D182E | A159V | L335M | D350Y | | | |
| TrzN cc2.29 | Y67F | A159V | L243P | | | | |
| TrzN cc2.30 | D38N | A159V | | | | | |
| TrzN cc2.31 | M163I | A159V | Y195F | D350Y | | | |
| TrzN cc2.32 | F156L | P210A | D350Y | | | | |
| TrzN cc2.33 | Y67F | D350Y | | | | | |
| TrzN cc2.34 | A159V | D350Y | | | | | |
| TrzN cc2.35 | Y67F | D99G | A159V | V213I | L243P | L335M | |
| TrzN cc2.36 | E109A | A159V | L314P | V320E | V399A | V429D | |
| TrzN cc2.37 | A159V | L335M | | | | | |
| TrzN_cc3.1 | Y67F | A159V | L335M | D350Y | | | |
| TrzN_cc3.2 | D38N | L131P | A159V | | | | |
| TrzN_cc3.3 | T91S | L131P | A159V | A294T | D350Y | R365H | L396M |
| TrzN_cc3.4 | R183H | P210A | D350Y | | | | |
| TrzN_cc3.5 | Y67F | A159V | D350Y | | | | |
| TrzN_cc3.6 | A159V | P210A | A294T | D350N | | | |
| TrzN_cc3.7 | A159V | P210A | A294T | D350N | | | |
| TrzN_cc3.8 | Y67F | A159V | D350N | | | | |
| TrzN_cc3.9 | A159V | L335M | D350Y | | | | |
| TrzN_cc3.10 | P210A | A294T | D350Y | | | | |
| TrzN_cc3.11 | T91S | A159V | A294T | | | | |
| TrzN_cc3.12 | P210A | A294T | L335M | | | | |
| TrzN_cc3.13 | Y67F | L335M | | | | | |

Interestingly, mutations that both resulted in an amino acid substitution and those that did not appear to influence the rate of clearing, suggesting that there are transcriptional or translational determinants of total TrzN activity in addition to factors affecting protein folding and stability. This is clearly demonstrated by the effect of the trzNco to trzN L1 mutation (T468C) which made no change to the protein sequence, but rather altered the phenylalanyl codon used to encode F156 (from TTT to TTC). Despite their identical products, trzN L1 clearly out performed its parental gene (Table 3).

### Site-Saturation Mutagenesis

Amino acids that were altered more than once during the first, second or third iteration of the random mutagenesis program, or were strongly selected for in the second and third iterations of random mutagenesis, or were present in one round of random mutagenesis and combined with other mutations in the parent template of the second and third iterations of random mutagenesis as independent second-site mutations, were deemed to be important determinants of the improved atrazine dechlorinase activities of the trzNco mutants. To more fully explore the sequence space at these positions, including replacement by amino acids unlikely to be introduced by random mutagenesis, site-saturation libraries were prepared for each of these sites (codons 67, 91, 131, 159, 161, 210, 243, 246, 294, 335 and 350; Table 6).

TABLE 6
The underlined amino acids were identified by
random mutagenesis; nonunderlined amino acids were
identified by site saturation mutagenesis only.

| Wild-type | Improving amino acid substitutions |
|---|---|
| Y67(TAT) | F(TTT) |
| T91(ACT) | S(TCT) |
| L131(CTG) | P(CCC), N(AAC), T(ACC), D(GAC), V(GTG), |
| | G(GGC), C(TGC), S(AGC), Q(CAG), H(CAC), |
| | Y(TAC), I(ATC) |
| A159(GCG) | V(GTG), T(ACG) |
| S161(AGC) | G(GGC) |
| P210(CCG) | A(GCG) |
| L243(CTG) | P(CCG), G(GGC) |
| G246(GGT) | A(GCG), S(AGC), E(GAG), K(AAG), V(GTG), |
| | D(GAC) |
| A294(GCG) | T(ACG), S(TCT), L(CTG) |
| L335(CTG) | M(ATG) |
| D350(GAC) | Y(TAC), N(AAC), F(TTC), R(CGC), H(CAC) |

Amino acid substitution by a wide spectrum of alternatives at some sites resulted in an increase in clearing rates compared with the parental gene, whilst others tolerated only a fairly conservative change to only one alternative amino acid. Substituting L131, D350 and G246 for twelve (P, N, T, D, V, G, C, S, Q, H, Y or I), six (D, S, E, K, V or A) and five (Y, N, F, K or H) alternative amino acids respectively resulted in increased dechlorinase activity compared with the wild-type enzyme. A294 could be substituted with three alternate amino acids (T, S or L) yielding greater atrazine dechlorinase activity, whilst A159 and L243 could only be successfully be substituted with two alternatives each (V or T, and P or G, respectively). At five of the positions, only one possible substitution yielded improved activities (Y67F, T91S, S161G, P210A and L335M) (Table 6). We also screened 100 colonies from a D38X site stauration library in a L131P A159V backgorund. We obtained 5 colonies with apparently improved clearing phenotypes. The only substitution to give such a phenotype was D38N.

### Characterisation of the Mutations in Variant TrzN cc3.2

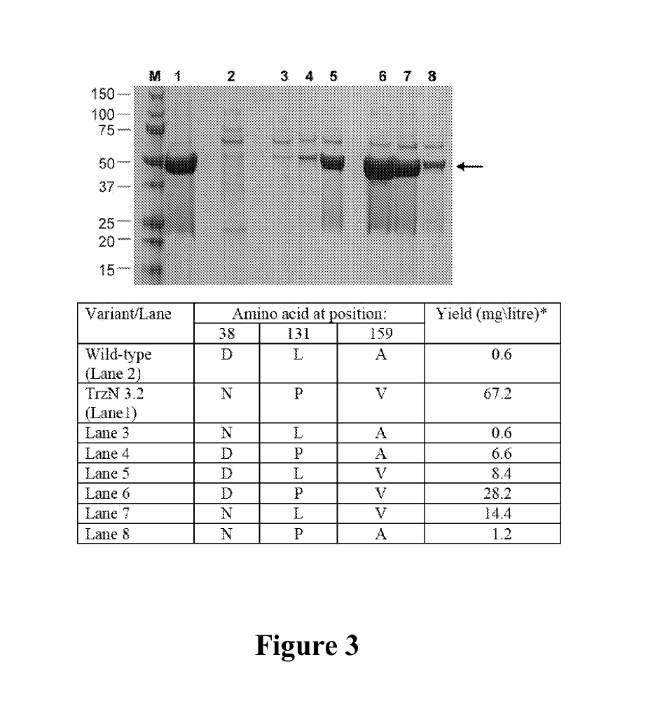

SDS-PAGE and kinetic analysis demonstrated that the vast majority of the improvement in activity was a result of increased expression. Indeed, the expression incrementally increased in each round of mutagenesis, and the most active mutant obtained in the third round contained three amino acid substitutions (D38N, L131P and A159V) (FIG. 3). There were a number of silent mutations in trzN, which may have contributed to the increased expression through changes in tRNA usage, mRNA stability or secondary structure effects upon translation efficiency. The contribution of each amino acid change to the increase in solubility was assessed by introducing them into the TrzN wild-type background individually, and introducing the reversion mutations into the mutant individually. The presence of the A159V substitution, the first mutation introduced during mutagenesis, enhanced soluble expression to 12.5% (8.4 mg/L) of that of the final variant. Introduction of the second substitution (L131P) into the A159V background enhanced soluble expression to 42% (28 mg/L) of that of the final variant, and introduction of the third substitution (D38N) into the L131P, A159V background increased the soluble expression to 67.2 mg/L (FIG. 3). In the wild-type TrzN background the L131P mutation had a similar effect upon solubility as the A159V substitution (6.6 mg/L yield). Therefore, the effect of combining the mutations is synergistic, rather than additive. Indeed the D38N, L131 variant was considerably less soluble than the L131P variant, whilst the D38N variant was similar in yield to that previously reported for wild-type TrzN suggesting that the D38N substitution negatively affects enzyme yields, except in the presence of the A159V substitution.

### Substrate Range

Purification of TrzN cc3.2 by size exclusion chromatography revealed that it had a native molecular weigth of between 75 and 150 kDa, suggesting that it is a homodimer or homotrimer. This is in contrast to AtzA, which is a homohexamer, and previous reports that TrzN was a monomer (Shapir et al., 2006).

The purified enzyme was used to determine the substrate range of an iteration 3 TrzN variant. TrzN cc3.2 was able to hydrolyse atrazine, ametryn, propazine, prometryn, simazine, simetryn, ipazine, trietazine, and cyanozine (Table 7), which represent s-triazines with halogen and methylthiol leaving groups, N-ethyl, N-isolpropyl, N-diethyl and N-cyanodimethylmethyl alkyl side chains. As the chemistry of methylthiol and methoxy leaving groups is highly similar, it is expected that TrzN cc3.2 retains the previously reported activity against methoxy-s-traizines (atraton, for example) (Shapir et al., 2005b).

TABLE 7
Specific activity data for purified TrzN cc3.2 versus a range of
triazines. Specific activity reported was measured at 100 μM
substrate and 41 nM TrzN cc3.2.

| | | | | Relative | Relative |
|---|---|---|---|---|---|
| | | | | Activity | Activity |
| | Leaving | N-alkyl | N-alkyl | in TrzN | in |
| Substrate | group | chain 1 | chain 2 | cc3.2 | TrzN* |
| Atrazine | chloride | ethyl | isopropyl | 1.36 | 0.16 |
| Ametryn | methylthiol | ethyl | isopropyl | 1 | 1 |
| Propazine | chloride | isopropyl | isopropyl | 0.86 | ND |
| Prometryn | methylthiol | isopropyl | isopropyl | 1 | ND |
| Simazine | chloride | ethyl | ethyl | 1.36 | 0.41 |
| Simetryn | methylthiol | ethyl | ethyl | 0.57 | 0.14 |
| Ipazine | chloride | isopropyl | diethyl | 0.07 | ND |
| Trietazine | chloride | ethyl | diethyl | 0.04 | ND |
| Cyanozine | chloride | ethyl | cyanodi- | 0.43 | ND |
| | | | methylmethyl | | |

*from Shapir et al. (2006).
ND: not determined.

### Host and Vector Range

The entire coding region for both TrzNcc3.2 and wild-type TrzN were moved from the inducible, high-level expression vector pETcc2 into a low-level, constitutive expression vector (pCS150, Scott et al., 2009). The resultant vectors were used to transform E. coli JM109, DH10β, and BL21 λDE3 cells. The six resultant strains were tested for their rates of clearing using the atrazine clearing plate assay. In each case the TrzNcc3.2 expressing strain cleared within two days at 37° C., whilst the strains expressing wild-type TrzN did not clear after 12 days at 37° C. This demonstrates that the improvements to TrzN expression were neither plasmid nor strain dependent.

### Example 2

### Structure of TrzN Mutants with Enhanced Activity

### Methods and Materials

### Structure Solution

Data collection statistics have been reported elsewhere (Jackson et al., 2006). Phase determination using single-wavelength anomalous diffraction (SAD) (Dauter et al., 2002) from the active site metals of metallo-enzymes has been previously reported (Liu et al., 2005). Because previous work suggested that the asymmetric unit contained a TrzN dimer (Jackson et al., 2006), SHELXD (Schneider et al., 2002), as implemented in the CCP4 suite of programs (Collaborative-Computational-Research-Project-4, 1994), was used to locate the positions of the four Zn²⁺ ion sites in an anomalous difference Patterson synthesis using the SAD data collected at 1.28269 Å. MLPHARE (Otwinowski, 1991) was subsequently used to refine the occupancy of the four sites and obtain initial phases with a phasing power of 2.43. SHELXE was used for density modification and phase improvement (Sheldrick et al., 2002); the high solvent content (79%) undoubtedly contributed to the quality of the phases (Terwilliger, 2001).

### Model Building and Refinement

The initial phases were used to perform automated model building with ARP-wARP (Perrakis et al., 2001). This produced an initial model with R_{free} of 38.0%. Several rounds of interactive model building were then carried out using COOT (Emsley and Cowtan, 2004), followed by structure idealisation as implemented in REFMAC v5.0 (Murshudov et al., 1997), after which R_{free} was 28.7%. Restrained refinement and the addition of water molecules reduced R_{free} to 23.5%. The B-factors were then set to 20, and 10 rounds of TLS refinement (Winn et al., 2001), using three rigid bodies comprising residues 1-195, 196-255 and 256-271 of each chain, followed by 3 rounds of maximum likelihood refinement, further lowered R_{free} to 20.0%, then 19.6%. TLSANL (Howlin et al., 1993) and ANISOANL (Winn, 2001) were used to analyse the data produced by TLS refinement. The libration tensors produced by TLSANL were visualised using RIBBONS (Carson, 1991), using a scale factor of 1.5. Geometric validation of the structures was made using RAMPAGE (Lovell et al., 2003), which indicated all residues were in favourable or allowed regions; PROCHECK (Laskowski et al., 1993), which indicated all stereochemical parameters were better than or inside normal limits; and SFCHECK (Vaguine et al., 1999), which gave an overall error in coordinates by Luzatti plot of 0.45 A².

### Results

The structure of TrzNcc3.2 was solved by molecular replacement using the 27% identical structure 2PAJ, from an environmental sample of the Sargasso Sea (Argawal, R. et al., unpublished). The correct solution was only found after extenisve ‘pruning’ of the search model, and even then only one molecule of the dimer could be found, which was the second best hit using the program PHASER (McCoy et al., 2007). The second molecule in the dimer was subsequently found using the program MOLREP (Vagin and Teplyakov, 2000). An initial R_{free} for the model of 53.4% was reduced to 21.1% after extensive model building and refinement.

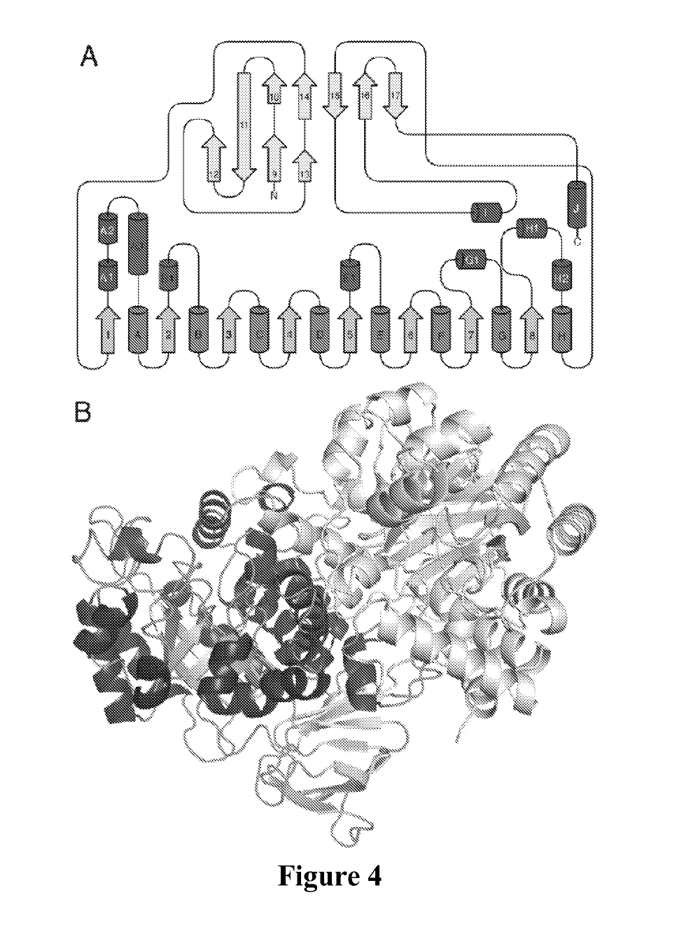

There is one 99.8 kDa homodimer in the crystallographic asymmetric unit, containing two, effectively identical, 49.6 kDa subunits of TrzN (FIG. 4), related by a non-crystallographic two-fold axis at (θ, φ)=(93, 90°), in polar angles. Despite low sequence similarity to known structures, TrzNcc3.2 adopts a (β/α)₈ barrel structural fold and belongs to the a large and functionally diverse metal-dependent amidohydrolase superfamily. The most similar structures in the protein data bank (PDB) are mostly of unknown function, such as 2PAJ. However, of those that are functionally annotated, the closest relatives are Tm0938, the 5-methylthioadenosine/S-adenosylhomocysteine/adenosine deaminase from Thermotoga maritima with 21% sequence identity, and main-chain r.m.s.d of 2.2 Å (Hermann et al., 2007), human guanine deaminase (2uz9; Moche, M et al., unpublished) with 19% sequence identity, and main-chain r.m.s.d of 2.5 Å, and an imidazolone propionase from Bacilus subtilis, with 19% sequence identity, and main-chain r.m.s.d of 2.9 Å (Yu et al., 2006).

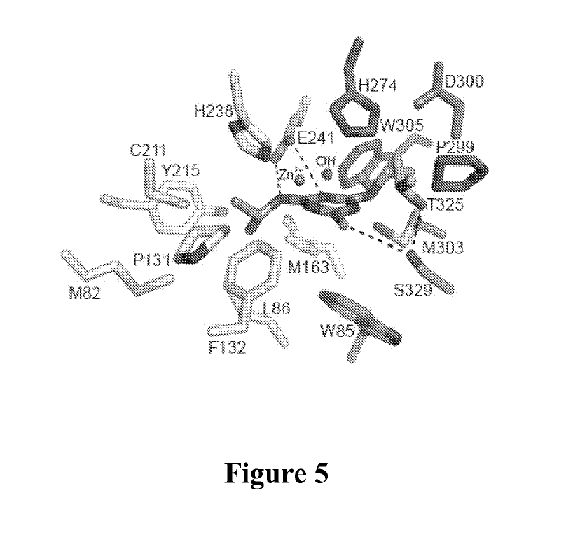

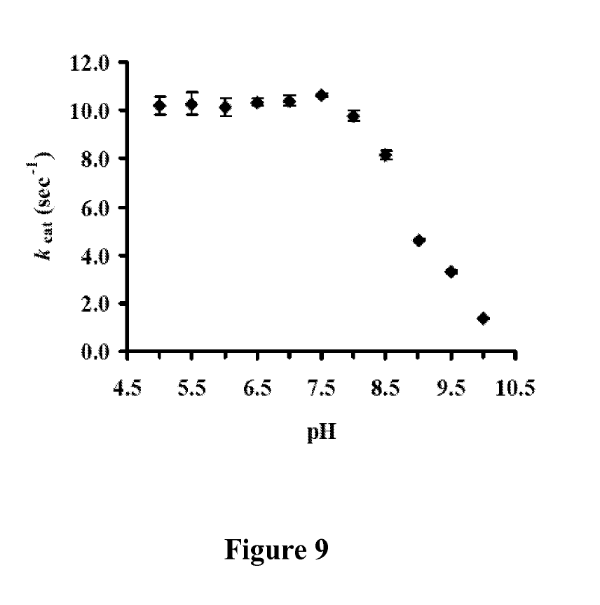

The active site cavity of TrzN (FIG. 5) is located at the centre of the catalytic domain of each subunit. A diagram of the active site architecture, with the substrate atrazine docked, in shown in FIG. 4. Three conserved histidines of the amidohydrolase motif, located on strands one (H63, H65) and five (H238), in addition to a glutamine residue from strand two and the putative water/hydroxide nucleophile constitute the metal ligands. The active site Zn²⁺ metal ion is coordinated in trigonal bipyramidal geometry, with H63, H65, and H238 comprising the equatorial ligands, and Q142 and the water molecule the axial ligands. The bond-length to Q129 (3.5 Å) is longer than expected but could be significantly shortened through minimal movement of this residue. As the Zn²⁺ metal ion is only bound at low occupancy (ca. 20%), we suggest the fully occupied active site may have a stronger Q129-Zn²⁺ interaction. Since the low occupancy of the metal made its assignment based on electron density ambiguous, anomalous data collected at the Zn K-edge was used to calculate a Bijvoet difference Fourier map of the active site of Zn²⁺-TrzN, which has been shown to be effective in identifying active site metal ions in metalloenzymes. This map unambiguously showed Zn²⁺ to be bound in the active site at the expected position. The nucleophilic water is shown to be additionally hydrogen bonded to H274, which is a conserved residue of the amidohydrolase motif and is in turn hydrogen bonded to D300 to form a H-D catalytic dyad. Kinetic analysis at different pH values suggests the nucleophile has a pKₐ of approximately 8 (FIG. 9), consistent with further activation of the Zn²⁺-OH⁻, which has a pKₐ value in solution of 8.4.

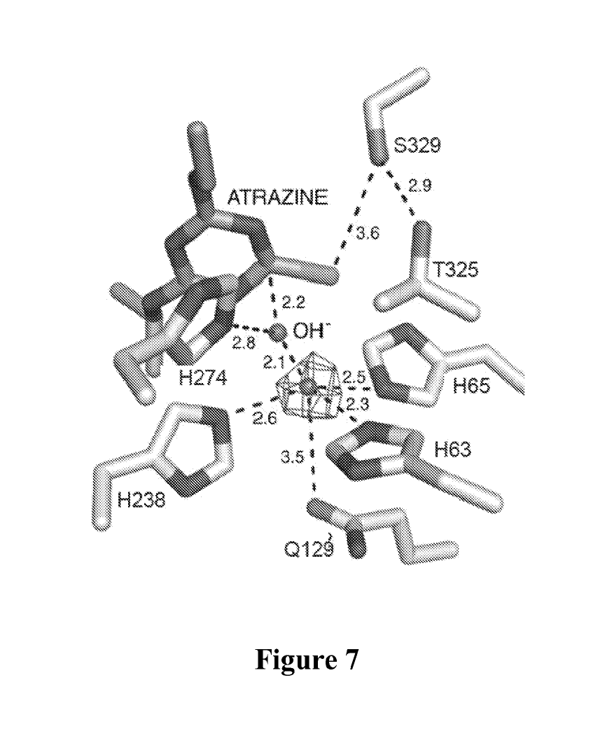

Although superficially similar to the active sites of other members of the metal ion-dependent amidohydrolase family, there is one major difference: the conserved aspartic acid metal ligand present in all other amidohydrolase structures is replaced by a threonine residue that is unable to serve as a metal ion ligand. The aspartic acid has apparently been functionally replaced in a non-analogous position by Q129, which restores the triginal bipyramidal coordination geometry of the metal ion. Replacement of a charge metal ion ligand by a polar ligand has the potential to reduce metal ion affinity (FIG. 7). The active site of the metallo-phosphodiesterase from Enterobacter aerogenes is informative in this respect; the coordination spheres of the two metal ions in the active site differs in the replacement of an aspartic acid in the α-site by an asparagine in the β-position, which was shown to result in a marked reduction in metal ion affinity for the β-site. Indeed, it appears that TrzN also has relatively weak affinity for metal ions; although excess Zn²⁺ was added to the growth media, and no metal chelators were used during purification or crystallization, the crystal structure has very low occupancy of zinc (see above).

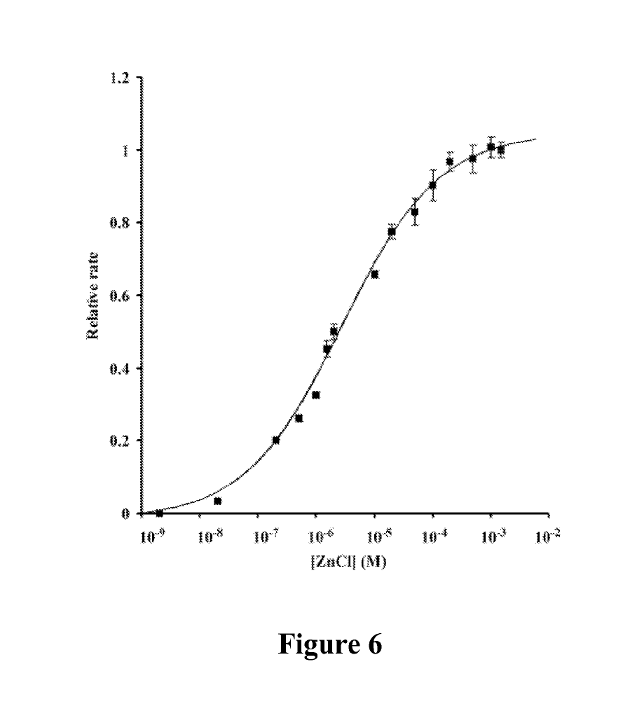

The dissociation constant for Zn²⁺ was determined to be 2.6 μM (FIG. 6), between five and six orders of magnitude higher than that determined for many Zn²⁺-containing enzymes, which often have K_{d} for zinc in the low picomolar range (carboninc anhydrase K_{d}=4 pM; Glyoxylase I, K_{d}=27 pM; dipeptidyl peptidase III, K_{d}=17 pM; carboxypeptidase A, K_{d}=1.6 pM; and superoxide dismutase, K_{d}=10 pM.

In order to characterise the substrate-binding pocket of TrzN, atrazine was manually modelled into the active site (FIGS. 5 and 7). The cavity can be divided into four sections: the isopropyl and ethyl side-chain pockets, a residue that interacts through π-π stacking with the aromatic ring of atrazine and residues that hydrogen-bond with the substrate and/or product. In addition to the metal ion ligand H238, the isopropyl side-chain pocket is formed by the side-chains of M82, L86, P131, F132, M163, C198 and Y215. The ethyl side-chain pocket is formed by the nucleophile ligand H274 as well as the side-chains of four residues, P299, D300, M303 and W305. At the ‘base’ of the pocket, W85 forms π-π stacking interactions with the aromatic ring of atrazine, which will stabilize both binding and the negative charge that will develop during the transition state. Finally, E241 is positioned to form hydrogen bonds with atrazine as the oxygen atoms of the carboxyl group is 3.3 Å from the NH groups of the isopropyl side-chain, while the S328-T325 dyad, linked by a 2.9 Å hydrogen bond will be able to interact with the chloride ion produced through hydrolysis, stabilizing the negative charge that will develop on the tetrahedral intermediate.

Interestingly, recent work on a natural TrzN variant from Nocardioides sp. strain AN3 identified an E241Q mutation that abolished catalytic activity towards substrates with poor leaving groups, such as ametryn (Yamazaki et al., 2008). The present inventors tested these effects in our system and also found a change in activity as a result of this mutation, but suggest it is principally a result of a reduction in substrate turnover (k_{cat}) rather than binding (Kₘ) (Table 8). The observation that the turnover rate is affected is also consistent with the Q241 mutant having reduced activity with substrates with poor leaving groups, suggesting that the mutation reduces the ability of the enzyme to effectively lower the activation energy to the reaction. In the same report positions 214 and 215 were identified as also affecting the catalysis of poor substrates. Again we show this to be due to a reduction in k_{cat} (Table 8). Overall, the complementary nature of the substrate binding cavity is striking, both in terms of shape and hydrophobicity. Excluding metal ligands and residues involved in hydrogen bonding to the substrate/product, 11/12 residues are hydrophobic, which will promote tight and energetically favourable binding and desolvation of the equally hydrophobic atrazine substrate.

TABLE 8
Effects of amino acid substitutions at positions 214, 215 and 241 in TrzN
cc3.2.

| | Amino acid | | | Atrazine | | Ametryn | |
|---|---|---|---|---|---|---|---|
| | at position | | | | Kₘ | | Kₘ |
| | 214 | 215 | 241 | k_{cat} (sec⁻¹) | (μM) | k_{cat} (sec⁻¹) | (μM)00 |
| TrzN 3.2 | P | Y | E | 3.5 | 49 | 10.5 | 43 |
| | T | Y | E | 1.8 | 53 | 0.4 | 49 |
| | T | H | E | 1.3 | 59 | 0.3 | 61 |
| | P | Y | Q | 12.1 | 61 | ND | ND |
| | P | H | Q | 3.4 | 58 | ND | ND |

ND; not detected

It is interesting that while the active site seems ideally suited to atrazine, it is completely closed to the bulk solvent, i.e. there is no way in which atrazine would be able to enter this substrate binding pocket. This is therefore an instance where conformational dynamics can be suggested to be integral to substrate turnover with some confidence. The closed active site cleft is shown in FIGS. 5 and 7, in which a network of hydrophobic residues (L86, M92, L172, Y215, L243, M247, M303 and W305) interact at the active site entrance, effectively ‘zipping’ it closed. The ‘lock’ is L172 of loop 3, which it located at the top of the network and will effectively hold the other residues in place. A plausible mechanism by which the active site opens principally concerns L185, which is located at the apex of a particularly mobile loop, in which the average B-factor, relative to the rest of the protein, is very high, consistent with partial occupancy/high mobility. Conformational change in this loop is likely to free the two sides of the active site cleft to separate in a ‘breathing’ motion that has been observed in similar enzymes (Jackson et al., 2007) and allow substrate to enter and product to depart. Indeed, the role of conformational fluctuations of surface loops in members of this family has recently been addressed and could serve to module the turnover rate by switching the enzyme between conformational substrates optimised for catalysis (closed) and diffusion (open).

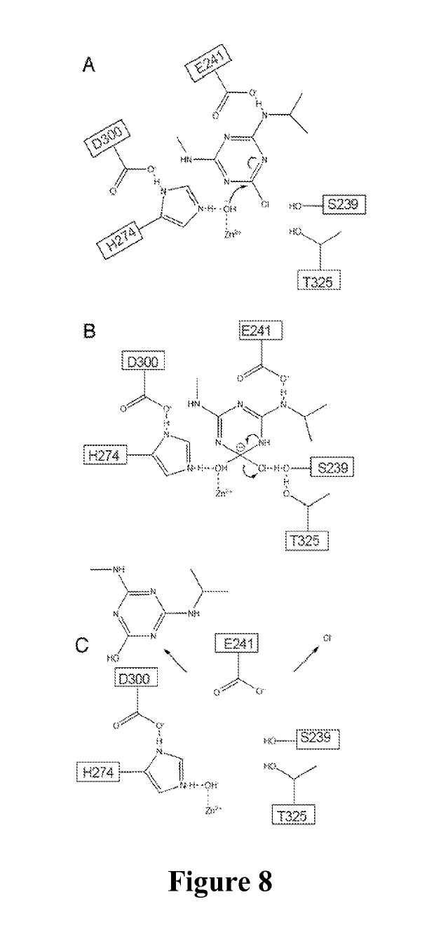

The structure, substrate docking and kinetics of TrzN allow a catalytic mechanism to be proposed as outlined in FIG. 8. The reaction can be broken down into four steps (i) substrate binding and nucleophile generation, (ii) nucleophilic attack, (iii) decomposition of the tetrahedral intermediate, (iv) product release. There are two catalytic dyads present in the active site of TrzN that are likely to play important roles in catalysis. The first of these, the D300-H274 dyad appears essential for generation of the nucleophilic hydroxide, in concert with the active site zinc metal ion. The active site Zn²⁺ ion will act as a Lewis acid, lowering the pKₐ of the bound water, while H274, positioned and stabilised by D300, will contribute to its deprotonation to form a nucleophilic hydroxide. Substrate binding will principally involve π-π stacking interactions with W85 and hydrogen bonding with E241. The loss of activity towards poor leaving groups (Yamazaki et al., 2008) and the reduction in the k_{cat} number upon mutagenesis of this residue to Q241 (Table 8) suggests that the electrostatic interaction with E241 may serve to activate the substrate in addition to optimising orientation. After substrate binding and nucleophile generation, nucleophilic attack will occur at the C4 carbon, resulting in the formation of a tetrahedral intermediate (the alignment makes SN2 displacement impossible), with a delocalised negative charge dispersed across the new hydroxyl group, the chlorine atom, and within the aromatic triazine ring in the transition state. This charge will be stabilised by the D300-H274 dyad at the hydroxyl group, the 5329-T325 dyad at the chlorine atom, and by the π-π stacking with W85 at the aromatic ring. Decomposition of this intermediate will yield the dechlorinated product hydroxyatrazine in addition to a free chloride. Product release will then require conformational change in the enzyme and opening of the active site cleft.

In conclusion, the following residues may be changed to alter the specific activity, catalytic constant (k_{cat}), substrate specificity (Kₘ), stability and/or second order rate constant (k_{cat}/Kₘ): M82, W85, L86, M92, P131, M163, L172, C211, Y215, H238, E241, L243, M247 H274, P299, D300 M303, W305, T325 or S329.

### Example 3

### Field Study

### Methods and Materials

### Preparation of TrzNcc3.2-Containing Homogenate

Clarified bacterial homogenate containing active TrzNcc3.2 were prepared from a 2 litre ferment of BL21 λDE3 expressing TrzNcc3.2, grown on a minimal medium (10.6 g/L KH₂PO₄, 4 g/L (NH₄)₂HPO₄, 1.7 g/L citric acid monohydrate, 31.3 mL/L glycerol). After autoclaving 10 mL/L of PTM4 salts (0.2 g/L D-biotin, 2.0 g/L CuSO₄.5H₂O, 0.08 g/L NaI, 3.0 g/L MnSO₄.H₂O, 0.2 g/L Na₂MoO4, 0.02 g/L Boric acid, 0.5 g/L CoCl₂.6H₂O, 7.0 g/L ZnCl₂, 22.0 g/L FeSO₄.7H₂O, 0.5 g/L CaSO₄, 1 mL/L H₂SO₄) and 0.6 g/L MgSO₄ was added. The fermentation was fed with glycerol, supplemented with 150 mg/L ampicillin and 331 mg/L thiamine, and induced with 11.9 mg/L IPTG. The ferment yielded ca. 240 g wet weight of cell pellet (OD₆₀₀=122). The cells were suspended in 5.2 g/L MOPS pH 6.9, then passed through a homogeniser 3 times and clarified by centrifugation. The clarified lysate was passed through a 0.22 μM filter to remove intact cells and DNaseI was used to remove intact DNA. Enzymatic activity was determined (258±19 mg of atrazine/mg of lysate/minute) using both the UV absorbance method described by de Souza et al. (1996) and the colorimetric method described in Scott et al. (2009). The homogenate was stored at −80° C. and thawed at 4° C. when required.

### Preparation of Test Dam

A ca. 1.5 mL holding dam at a sugar cane farm near Clare (Lat. 19:48, Long. 147:14) in the wet/dry tropics of Queensland, Australia, was filled with headwater from irrigation of a field pre-treated with the recommended dose of atrazine (3.3 kg per hectre). 240 g of bacterial homogenate was suspended in 20 litres of water, and applied by hand by spreading evenly across the surface of the holding dam. Duplicate 1 litre samples were taken before the dam was filled with atrazine-contaminated runoff water, before the enzyme was added, and at time intervals after the addition of the enzyme. Samples were stored immediately on ice to stop the enzymatic reaction. Samples were frozen after no more than 4 hours on ice.

### Determination of Atrazine Concentration

Atrazine concentrations were determined at two independent laboratories; Queensland Health Forensic and Scientific Services (QHFSS), by the LCMSMS method described in Lewis et al. (2009), modified to use direct injection; and by CSIRO Entomology by the following LCMS method. Briefly, 100 mL samples were acidified with HCl to pH 2.8, then the atrazine in the samples was concentrated 1000-fold by solid phase extraction using preconditioned Oasis SPE Max Cartridges (Waters, USA), and eluted in 3 mL of MeOH (with ammonia). Samples were subsequently dried and dissolved in 100 μl of MeOH. Samples were separated by HPLC and assayed for atrazine concentrations by measuring the absorbance at 265 nm, and the analyte peak area calculated using Analyst software. Replicate samples were within 10% agreement. The identity of the HPLC peak was confirmed by mass spectrum analysis, whereby atrazine ions 216 m/z were extracted on an Agilent ToF-MSD.

### Results

A ca. 1.5 mL holding dam at a sugar cane farm near Clare (Lat. 19:48, Long. 147:14) in the wet/dry tropics of Queensland, Australia, was filled with headwater from irrigation of a field pre-treated with the recommended dose of atrazine (3.3 kg per hectre). 240 g of bacterial homogenate producing TrzNcc3.2 was suspended in 20 litres of water, and applied by hand by spreading evenly across the surface of the holding dam. Duplicate 1 litre samples were taken before the dam was filled with atrazine-contaminated runoff water, before the enzyme was added and at time intervals after the addition of the enzyme. Samples were stored immediately on ice to stop the enzymatic reaction. Samples were frozen after no more than 4 hours on ice.

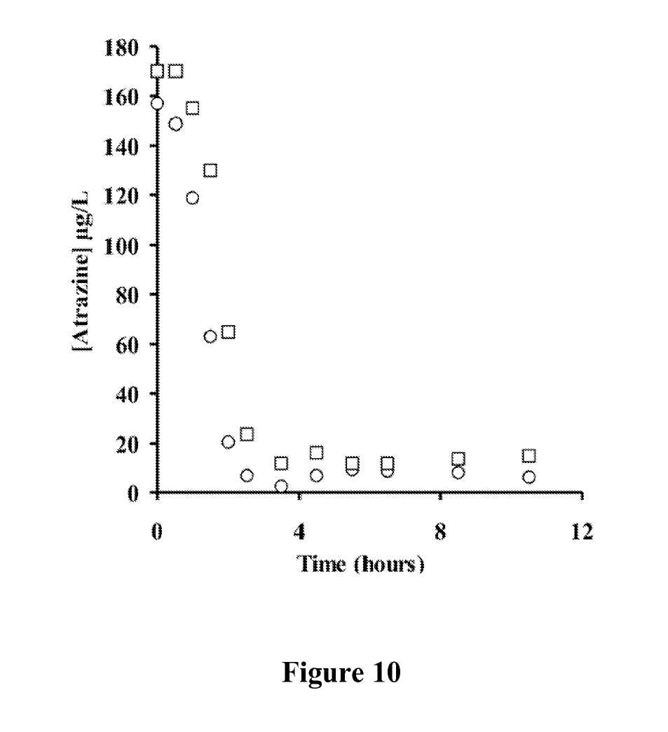

The water in the holding dam contained 8-12 μg/L atrazine before the irrigation tailwater was collected (data not shown). After filling with irrigation tailwater the atrazine concentration rose to 157-170 μg/L (FIG. 10). There was a lag in the rate of atrazine depletion after addition of the enzyme, which was most likely attributable to the rate at which the enzyme mixed with the water in the holding dam. The duration of the “mixing phase” for enzyme applied in this manner is almost certainly dependent on the volume and surface area:volume ratio of the water body to be remediated; i.e. larger bodies and those with low surface area: volume ratios would require a longer mixing phase.

Notwithstanding the lag during the mixing phase, the addition of the enzyme led to >90% depletion in the concentration of atrazine in the first four hours after addition. This result indicate that a TrzN-based bioremediant for triazines is technically feasible.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The present application claims priority from U.S. 61/094,044, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Abdullah et al. (1986) Biotechnology 4:1087.
Alcalde et al. (2007) Biocatal. Biotransform. 25:113-113.
Belluck et al. (1991) Acs Symposium Series 459:254-273.
Boundy-Mills et al. (1997) Appl. Environ. Microbiol. 63:916-923.
Capecchi (1980) Cell 22:479-488.
Carson (1991) Journal of Applied Crystallography 24: 958-61.
Cheng et al. (1996) Plant Cell Rep. 15:653-657.
Cheng et al. (2005) Appl. Environ. Microbiol. 71:4437-4445.
Clapp (1993) Clin. Perinatol. 20:155-168.
Curiel et al. (1992) Hum. Gen. Ther. 3:147-154.
Dauter et al. (2002) Acta Crystallogr. D 58: 494-506.
de Souza et al. (1995) Appl. Environ. Microbiol. 61:3373-3378.
de Souza et al. (1996) J. Bacteriol. 178:4894-4900.
Eglitis et al. (1988) Biotechniques 6:608-614.
Emsley & Cowtan (2004) Acta Crystallogr. D 60: 2126-32.
Fruchey et al. (2003) Appl. Environ. Microbiol. 69:3653-3657.
Fujimura et al. (1985) Plant Tissue Culture Letters 2:74.
Gavrilescu (2005) Eng. Life Sci. 5, 497-526.
Gordon et al. (1999) Chemical-Biological Interactions 14:463-470.
Graham et al. (1973) Virology 54:536-539.
Grant et al. (1995) Plant Cell Rep. 15:254-258.
Harayama (1998) Trends Biotechnol. 16:76-82.
Hayes et al. (2002) Proc. Natl. Acad. Sci. U.S.A. 99:5476-5480.
Hayes et al. (2003) Environ. Health Perspect. 111:568-575.
Hayes et al. (2006) Environ. Health Perspect. 114:134-141.
Hermann et al. (2007) Nature 448: 775-9.
Howlin et al. (1993) J. Appl. Crystallogr. 26: 622-24.
Huff (2002) Int. J. Occup. Environ. Health 8:249-270.
Huff and Sass (2007) Int. J. Occup. Environ. Health 13, 356-358.
Jackson et al. (2006) Biochem. J. 397: 501-8.
Jackson et al. (2007) J. Mol. Biol. 367: 1047-62.
Koziel et al. (1996) Plant Mol Biol 32:393-405.
Laskowski et al. (1993) J. Appl. Crystallogr. 26: 283-91.
LeJuene et al. (1998) Nature 395:27-28.
Lewis et al. Enviro. Poll. 157: 2470-2484.
Liu et al. (2005) Proc. Natl. Acad. Sci. U.S.A. 102: 11882-7.
Lovell et al. (2003) Proteins 50: 437-50.
Lu et al. (1993) J. Exp. Med. 178:2089-2096.
Mandelbaum et al. (1995) Appl. Environ. Microbiol. 61:1451-1457.
Martinez et al. (2001) J. Bacteriol. 183:5684-5697.
McCoy et al. (2007) J. Appl. Crystallogr. 40: 658-674.
Murshudov et al. (1997) Acta Crystallogr. D 53: 240-55.
Needleman and Wunsch (1970) J. Mol. Biol., 48:443-453.
Nishihara et al. (1998) Appl. Environ. Microbiol. 64:1694-1699.
Nishihara et al. (2000) Appl. Environ. Microbiol. 66:884-889.
Otwinowski (1991). Daresbury Study Weekend proceedings.
Parales et al. (2002) Appl. Environ. Microbiol. 68:4699-4709.
Perrakis et al. (2001) Acta Crystallogr. D 57: 1445-50.
Petrikovics et al. (2000a) Toxicology Science 57: 16-21.
Petrikovics et al. (2000b) Drug Delivery 7: 83-89.
Sadowsky et al. (1998) J. Bacteriol. 180:152-158.
Sajjaphan et al. (2004) Applied and Environmental Microbiology 70: 4402-4407.
Scott et al. (2009) Appl. Environ. Microbiol. 75: 2184-91.
Shapir et al. (2005a) Journal of Bacteriology 187: 3731-3738.
Shapir et al. (2005b) Applied and Environmental Microbiology 71: 2214-2220.
Shapir et al. (2006) Journal of Bacteriology 188: 5859-5864.
Sheldrick (2002). Kristallogr. 217: 644-50.
Sutherland et al. (2004) Clin. Exp. Pharmacol. Physiol. 31:817-821.
Terwilliger (2001) Acta Crystallogr. D 57: 1763-75.
Thurman and Meyer (1996) Herbicide metabolites in surface water and groundwater: Introduction and overview. in Herbicide Metabolites in Surface Water and Groundwater.
Tomlin (2006) The Pesticide Manual BCPB.
Toriyama et al. (1986) Theor. Appl. Genet. 205:34.
Vagin & Teplyakov (2000) Acta Crystallogr. D. Biol. Crystallogr. 56: 1622-4.
Vaguine et al. (1999) Acta. Crystallogr. D 55: 191-205.
van der Meer (2006) Front. Ecol. Environ. 4:35-42.
Wagner et al. (1992) Proc. Natl. Acad. Sci. USA 89:6099-6103.
Winn (2001) CCP4 Newsletter 39.
Winn et al. (2001) Acta Crystallogr. D 57: 122-33.
Yamazaki et al. (2008) FEMS Microbiol. Lett. 286: 171-7.
Yu et al. (2006) J. Biol. Chem. 281: 36929-36.

1-41. (canceled)

42. A substantially purified and/or recombinant polypeptide which hydrolyses s-triazine or diazine, wherein the polypeptide comprises an amino acid sequence as provided in SEQ ID NO:1 with an asparagine at amino acid number 38 of SEQ ID NO:1, a proline at amino acid number 131 of SEQ ID NO:1, and a valine at amino acid number 159 of SEQ ID NO:1.

43. The substantially purified and/or recombinant polypeptide of claim 42, wherein when produced in an E. coli cell, more of the polypeptide is produced than by an isogenic E. coli cell cultured under identical conditions comprising an exogenous polynucleotide encoding the amino acid sequence provided as SEQ ID NO:1.

44. An isolated and/or exogenous polynucleotide encoding a polypeptide which hydrolyses s-triazine or diazine, wherein the polypeptide comprises an amino acid sequence as provided in SEQ ID NO:1 with an asparagine at amino acid number 38 of SEQ ID NO:1, a proline at amino acid number 131 of SEQ ID NO:1, and a valine at amino acid number 159 of SEQ ID NO:1.

45. The isolated and/or exogenous polynucleotide of claim 44, wherein when expressed in an E. coli cell, more of the polypeptide is produced than by an isogenic E. coli cell cultured under identical conditions comprising an exogenous polynucleotide encoding the amino acid sequence provided as SEQ ID NO:1.

46. The polynucleotide of claim 44 which is operably linked to a promoter capable of directing expression of the polynucleotide in a cell.

47. A vector comprising the polynucleotide of claim 46.

48. A host cell comprising the polynucleotide of claim 46.

49. The host cell of claim 48 which is a bacterial cell, yeast cell or a plant cell.

50. An extract of the host cell of claim 49, wherein the extract comprises the polypeptide which hydrolyses s-triazine or diazine.

51. The extract of claim 50, wherein when the polypeptide of the extract is produced in an E. coli cell, more of the polypeptide is produced than by an isogenic E. coli cell cultured under identical conditions comprising an exogenous polynucleotide encoding the amino acid sequence provided as SEQ ID NO:1.

52. A composition comprising the polypeptide of claim 42, and one or more acceptable carriers.

53. A composition comprising the extract of claim 50, and one or more acceptable carriers.

54. A method for hydrolysing s-triazine or diazine, the method comprising contacting the s-triazine or diazine with the polypeptide of claim 42.

55. A method for hydrolysing s-triazine or diazine, the method comprising contacting the s-triazine or diazine with the extract of claim 50.

56. A method of producing a polypeptide which hydrolyses s-triazine or diazine, wherein the polypeptide comprises an amino acid sequence as provided in SEQ ID NO:1 with an asparagine at amino acid number 38 of SEQ ID NO:1, a proline at amino acid number 131 of SEQ ID NO:1, and a valine at amino acid number 159 of SEQ ID NO:1, the method comprising cultivating the host cell of claim 48 under conditions which allow expression of the polynucleotide encoding the polypeptide, and recovering the expressed polypeptide.

57. The method of claim 56, wherein when produced in an E. coli cell, more of the polypeptide is produced than by an isogenic E. coli cell cultured under identical conditions comprising an exogenous polynucleotide encoding the amino acid sequence provided as SEQ ID NO:1.

58. A method of producing a polypeptide which hydrolyses s-triazine or diazine, wherein the polypeptide comprises an amino acid sequence as provided in SEQ ID NO:1 with an asparagine at amino acid number 38 of SEQ ID NO:1, a proline at amino acid number 131 of SEQ ID NO:1, and a valine at amino acid number 159 of SEQ ID NO:1, the method comprising exposing the vector of claim 47 to conditions which allow expression of the polynucleotide encoding the polypeptide, and recovering the expressed polypeptide.

59. The method of claim 58, wherein when produced in an E. coli cell, more of the polypeptide is produced than by an isogenic E. coli cell cultured under identical conditions comprising an exogenous polynucleotide encoding the amino acid sequence provided as SEQ ID NO:1.
